(19) United States
(12) Patent Application Publication
(10) Pub. No.: US 2023/0150965 A1
(43) Pub. Date: May 18, 2023

(54) ISOTOPICALLY ENRICHED N-METHYL-1,3-BENZODIOXOLYLBUTANAMINE (MBDB) AND STEREOISOMERS THEREOF

(71) Applicant: Terran Biosciences, Inc., New York, NY (US)

(72) Inventors: Matthew DUNCTON, San Bruno, CA (US); Samuel CLARK, New York, NY (US)

(21) Appl. No.: 17/982,468

(22) Filed: Nov. 7, 2022

Related U.S. Application Data
(60) Provisional application No. 63/284,960, filed on Dec. 1, 2021.
(60) Provisional application No. 63/276,550, filed on Nov. 5, 2021.

(51) Int. Cl. C07D 317/58; A61P 25/22; A61P 25/28

(52) U.S. Cl. CPC C07D 317/58; A61P 25/22; A61P 25/28; C07B 2200/05

(57) ABSTRACT

Disclosed herein are isotopically enriched compounds and methods of using both isotopically and non-isotopically enriched compounds of the following formula

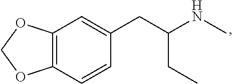

in the treatment of neurologic and brain disorders/conditions.

### CROSS REFERENCE TO RELATED APPLICATIONS

This application claims priority to and the benefit of U.S. Provisional Patent Application Nos. 63/276,550, filed Nov. 5, 2021, and 63/284,960, filed Dec. 1, 2021. The contents of each are incorporated by reference herein in their entirety for all purposes.

### BACKGROUND

Major depressive disorder and related neuropsychiatric diseases are among the leading causes of disability worldwide. Despite recent advances, there remains a need for new therapeutics to support treatment of debilitating neuropsychiatric diseases.

Recently, psychedelic compounds have received renewed interest for the treatment of depression and other disorders. For example, the Food and Drug Administration (FDA) recently approved the dissociative anesthetic ketamine for treatment-resistant depression, making it the first mechanistically distinct medicine to be introduced to psychiatry in nearly thirty years. Ketamine is a member of a class of compounds known as psychoplastogens. Psychoplastogens promote neuronal growth through a mechanism involving the activation of AMPA receptors, the tropomyosin receptor kinase B (TrkB), and the mammalian target of rapamycin (mTOR). As pyramidal neurons in the PFC exhibit top-down control over areas of the brain controlling motivation, fear, and reward, these effects support clinical development of psychoplastogenic compounds for their antidepressant, anxiolytic, and anti-addictive effects properties.

### 3,4-N-methyl-1,3-benzodioxolylbutanamine (MBDB)

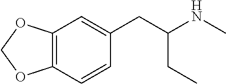

N-methyl-1,3-benzodioxolylbutanamine (MBDB) 1-(benzo[d][1,3]dioxol-5-yl)-N-methylbutan-2-amine is a synthetic analog of the psychedelic phenethylamine class of compounds. The present application addresses a currently unmet need of providing compounds with improved properties relative to MBDB.

### SUMMARY

The present disclosure relates to MBDB analog compounds for the treatment of neurological and psychiatric disorders. In one embodiment the compounds have improved efficacy, improved pharmacokinetic properties or both. In one embodiment the disclosed compounds are isotopically enriched at one or more position. In one aspect of the disclosed embodiments the compounds are represented by Formula I

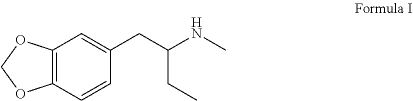

wherein an atom at one or more positions in Formula is enriched in an isotope.

The foregoing and other objects, features, and advantages of the invention will become more apparent from the following detailed description.

### BRIEF DESCRIPTION OF THE FIGURES

FIG. 1 illustrates the frequency of stretched attend postures (SAPs) after racemic MBDB, R-MBDB, and S-MBDB compared to vehicle and chlordiazepoxide control on the elevated zero maze.

### DETAILED DESCRIPTION

### General

Disclosed herein are phenethylamine analogs, in particular, isotopically labeled analogs, or isotopologues. The presently disclosed isotopologues are useful for the treatment of a variety of brain disorders and other conditions. Without limitation to any particular theory, it is believed that the present compounds increase neuronal plasticity, and increase at least one of translation, transcription, or secretion of neurotrophic factors. Moreover, by virtue of their isotopic enrichment, the presently disclosed compounds have improved pharmacokinetic and pharmacodynamic properties as compared to previously disclosed molecules. In certain embodiments the isotopic labels of the present compounds allow monitoring of its pharmacodynamic and ADME behavior following in vivo administration. In some embodiments, the isotopically enriched compounds described herein provide better therapeutic potential for neurological diseases than known compounds.

### Terms and Abbreviations

The term “isotopic enrichment factor” as used herein means the ratio between the isotopic abundance and the natural abundance of a specified isotope. It will be recognized that some variation of natural isotopic abundance occurs in a synthesized compound depending upon the origin of chemical materials used in the synthesis. Thus, a preparation of any compound will inherently contain small amounts of isotopologues, including deuterated isotopologues. The concentration of naturally abundant stable hydrogen isotopes, notwithstanding this variation, is small and immaterial as compared to the degree of stable isotopic substitution of compounds of this disclosure. In a compound of this disclosure, when a particular position is designated as having a particular isotope, such as deuterium, it is understood that the abundance of deuterium at that position is substantially greater than the natural abundance of deuterium, which is about 0.015% (on a mol/mol basis). A position designated as a particular isotope will have a minimum isotopic enrichment factor of at least 3000 (45% incorporation of the indicated isotope). Thus, isotopically enriched compounds disclosed herein having deuterium will have a minimum isotopic enrichment factor of at least 3000 (45% deuterium incorporation) at each atom designated as deuterium in the compound. Such compounds may be referred to herein as “deuterated” compounds.

In other embodiments, disclosed compounds have an isotopic enrichment factor for each designated atom of at least 3500 (52.5%). For example, for such disclosed compounds that are deuterium isotopologues, the compounds have an isotopic enrichment factor for each designated hydrogen atom of at least 3500 (52.5% deuterium incorporation at each designated atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As above, such compounds also are referred to as “deuterated” compounds.

In the compounds of this disclosure any atom not specifically designated as a particular isotope is meant to represent any stable isotope of that atom. Unless otherwise stated, when a position is designated specifically as “H”, the position is understood to have hydrogen at about its natural abundance isotopic composition.

The term “isotopologue” refers to a species that has the same chemical structure and formula as another compound, with the exception of the isotopic composition at one or more positions, e.g., H vs. D. Thus, isotopologues differ in their isotopic composition.

“Salt” refers to acid or base salts of the compounds used in the methods of the present invention, in particular pharmaceutically acceptable salts. Illustrative examples of pharmaceutically acceptable salts are mineral acid (hydrochloric acid, hydrobromic acid, phosphoric acid, and the like) salts, organic acid (fumaric acid, acetic acid, propionic acid, glutamic acid, citric acid, tartaric acid and the like) salts, quaternary ammonium (methyl iodide, ethyl iodide, and the like) salts. It is understood that the pharmaceutically acceptable salts are non-toxic. Additional suitable pharmaceutically acceptable salts are known to those of skill in the art. See, e.g., Remington: The Science and Practice of Pharmacy, volume I and volume II. (22^{nd} Ed., University of the Sciences, Philadelphia), which is incorporated herein by reference.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

“Pharmaceutically acceptable salt” refers to a compound in salt form, wherein the salt form is suitable for administration to a subject. Representative pharmaceutically acceptable salts include salts of acetic, ascorbic, benzenesulfonic, benzoic, camphorsulfonic, citric, ethanesulfonic, edisylic, fumaric, gentisic, gluconic, glucoronic, glutamic, hippuric, hydrobromic, hydrochloric, isethionic, lactic, lactobionic, maleic, malic, mandelic, methanesulfonic, mucic, naphthalenesulfonic, naphthalene-1,5-disulfonic, naphthalene-2,6-disulfonic, nicotinic, nitric, orotic, pamoic, pantothenic, phosphoric, succinic, sulfuric, tartaric, p-toluenesulfonic and xinafoic acid, and the like

“Pharmaceutically acceptable excipient” refers to a substance that aids the administration of an active agent to and absorption by a subject. Pharmaceutical excipients useful in the present invention include, but are not limited to, binders, fillers, disintegrants, lubricants, coatings, sweeteners, flavors and colors. One of skill in the art will recognize that other pharmaceutical excipients are useful in the present invention.

“Composition” refers to a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation.

“Isomers” refers to compounds with same chemical formula but different connectivity between the atoms in the molecule, leading to distinct chemical structures. Isomers include structural isomers and stereoisomers. Examples of structural isomers include, but are not limited to tautomers and regioisomers. Examples of stereoisomers include but are not limited to diastereomers and enantiomers.

“Administering” refers to any suitable mode of administration, including, oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Subject” refers to an animal, such as a mammal, including, but not limited to, primates (e.g., humans), cows, sheep, goats, horses, dogs, cats, rabbits, rats, mice and the like. In certain embodiments, the subject is a human subject.

“Therapeutically effective amount” or “therapeutically sufficient amount” or “effective or sufficient amount” refers to a dose that produces therapeutic effects for which it is administered.

The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

“Neuronal plasticity” refers to the ability of the brain to change its structure and/or function continuously throughout a subject's life. Examples of the changes to the brain include, but are not limited to, the ability to adapt or respond to internal and/or external stimuli, such as due to an injury, and the ability to produce new neurites, dendritic spines, and synapses.

“Brain disorder” refers to a neurological disorder which affects the brain's structure and function. Brain disorders can include, but are not limited to, Alzheimer's, Parkinson's disease, psychological disorder, depression, treatment resistant depression, addiction, anxiety, post-traumatic stress disorder, suicidal ideation, major depressive disorder, bipolar disorder, schizophrenia, stroke, traumatic brain injury, and substance use disorder.

“Combination therapy” refers to a method of treating a disease or disorder, wherein two or more different pharmaceutical agents are administered in overlapping regimens so that the subject is simultaneously exposed to both agents. For example, the compounds of the invention can be used in combination with other pharmaceutically active compounds. The compounds of the invention can be administered simultaneously (as a single preparation or separate preparation) or sequentially to the other drug therapy. In general, a combination therapy envisions administration of two or more drugs during a single cycle or course of therapy.

“Neurotrophic factors” refers to a family of soluble peptides or proteins which support the survival, growth, and differentiation of developing and mature neurons.

“Modulate” or “modulating” or “modulation” refers to an increase or decrease in the amount, quality, or effect of a particular activity, function or molecule. By way of illustration and not limitation, agonists, partial agonists, antagonists, and allosteric modulators (e.g., a positive allosteric modulator) of a G protein-coupled receptor (e.g., 5HT_{2A}) are modulators of the receptor.

“Agonism” refers to the activation of a receptor or enzyme by a modulator, or agonist, to produce a biological response.

“Agonist” refers to a modulator that binds to a receptor or enzyme and activates the receptor to produce a biological response. By way of example only, “5HT_{2A} agonist” can be used to refer to a compound that exhibits an EC₅₀ with respect to 5HT_{2A} activity of no more than about 100 mM. In some embodiments, the term “agonist” includes full agonists or partial agonists. “Full agonist” refers to a modulator that binds to and activates a receptor with the maximum response that an agonist can elicit at the receptor. “Partial agonist” refers to a modulator that binds to and activates a given receptor, but has partial efficacy, that is, less than the maximal response, at the receptor relative to a full agonist.

“Positive allosteric modulator” refers to a modulator that binds to a site distinct from the orthosteric binding site and enhances or amplifies the effect of an agonist.

“Antagonism” refers to the inactivation of a receptor or enzyme by a modulator, or antagonist. Antagonism of a receptor, for example, is when a molecule binds to the receptor and does not allow activity to occur.

“Antagonist” or “neutral antagonist” refers to a modulator that binds to a receptor or enzyme and blocks a biological response. An antagonist has no activity in the absence of an agonist or inverse agonist but can block the activity of either, causing no change in the biological response.

### Compounds:

Disclosed herein are isotopically enriched compounds of Formula I:

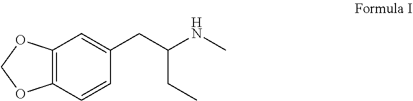

having improved properties relative to MBDB.

The present inventors observed that the metabolic properties of MBDB could be improved by isotopic enrichment, in particular, deuterium or tritium enrichment. In one embodiment of this approach, one attempts to slow the CYP-mediated metabolism of a drug or to reduce the formation of undesirable metabolites by replacing one or more protium (H) atoms with deuterium atoms. Deuterium is a safe, stable, non-radioactive isotope of hydrogen. Compared to protium, deuterium forms stronger bonds with carbon. In select cases, the increased bond strength imparted by deuterium can positively affect the pharmacokinetic properties of a drug, creating the potential for improved drug efficacy, safety, and/or tolerability. At the same time, because the size and shape of deuterium are essentially identical to those of protium, replacement of protium by deuterium would not be expected to affect the biochemical potency and selectivity of the drug as compared to the original chemical entity that contains only hydrogen. Tritium, ³H, forms still stronger bonds with carbon than deuterium. Thus, replacement of protium with tritium also can affect the pharmacokinetic properties of a molecule. Moreover, tritium is a beta emitter, meaning that enriching a molecule with tritium allows determination of pharmacokinetic and pharmacodynamic properties of the molecule to better understand its activity and ADME properties.

Accordingly, in certain embodiments, the present invention provides an isotopically enriched compound of Formula I:

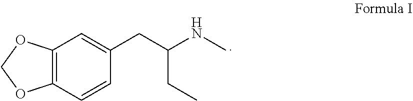

In one embodiment, the disclosed compounds of Formula I have Formula II

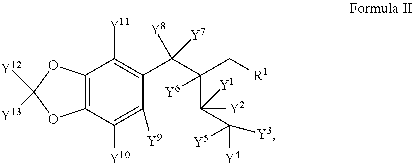

wherein at least one of R¹, Y¹, Y², Y³, Y⁴, Y⁵, Y⁶, Y⁷, Y⁸, Y⁹, Y¹⁰, Y¹¹, Y¹² and Y¹³ is enriched in at least one heavy isotope, selected from those such as ¹⁴C, tritium and deuterium.

With continued reference to Formula II, in one embodiment, R¹ is selected from CD₃, CD₂H, CDH₂, CT₃, CT₂H, CTH₂ and CH₃. In certain embodiments of Formula II, Y¹, Y², Y³, Y⁴, Y⁵, Y⁶, Y⁷, Y⁸, Y⁹, Y¹⁰, Y¹¹, Y¹² and Y¹³ are independently selected from protium, deuterium and tritium. In one such embodiment, at least one of R, Y¹, Y², Y³, Y⁴, Y⁵, Y⁶, Y⁷, Y⁸, Y⁹, Y¹⁰, Y¹¹, Y¹² and Y¹³ is enriched in deuterium.

Particular embodiments according to Formulas I and II have the structures:

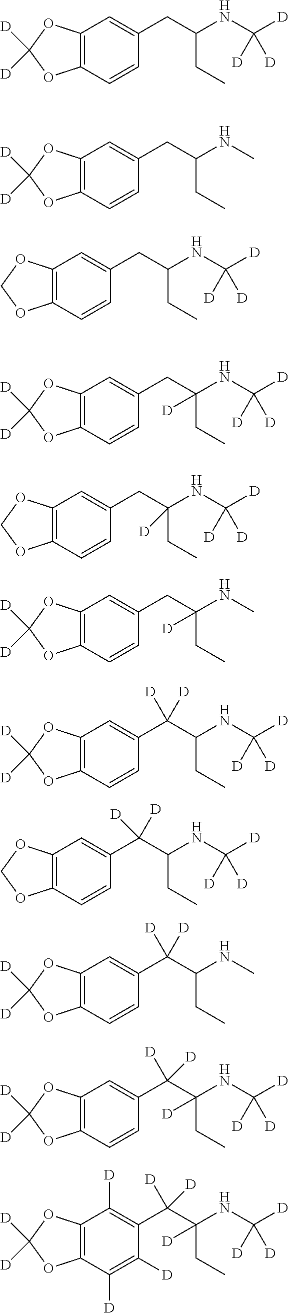

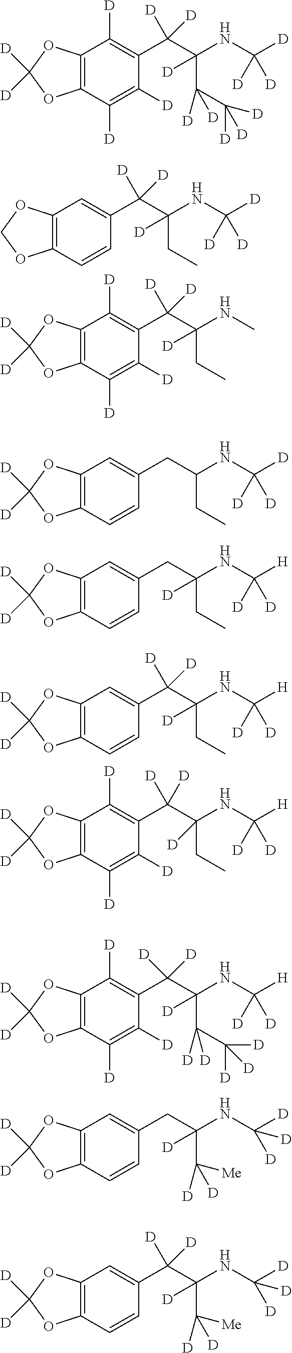

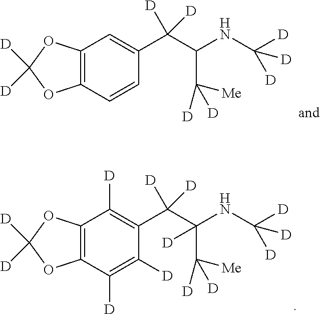 [SMILES: [2H]C([2H])([2H])NC(C([2H])([2H])C)C([2H])([2H])c1ccc2c(c1)OC([2H])([2H])O2 ; [2H]c1c([2H])c(C([2H])([2H])C([2H])(NC([2H])([2H])[2H])C([2H])([2H])C)c([2H])c2c1OC([2H])([2H])O2]

The disclosed compounds of Formula I, also are chiral, for example, the compounds may be the (S) enantiomer having the formula:

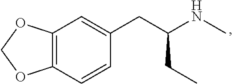

or the mirror image, (R) enantiomer having the formula:

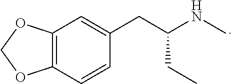

and may be present in racemic, optically enriched or optically pure forms.

Certain embodiments also may be represented by the formula

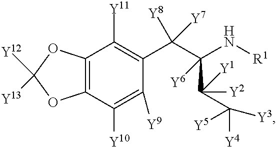

wherein at least one of R¹, Y¹, Y², Y³, Y⁴, Y⁵, Y⁶, Y⁷, Y⁸, Y⁹, Y¹⁰, Y¹¹, Y¹² and Y¹³ is enriched in at least one heavy isotope; or the formula

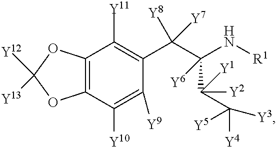

wherein at least one of R¹, Y¹, Y², Y³, Y⁴, Y⁵, Y⁶, Y⁷, Y⁸, Y⁹, Y¹⁰, Y¹¹, Y¹² and Y¹³ is enriched in at least one heavy isotope.

In more particular embodiments, the disclosed compounds are represented by a formula selected from:

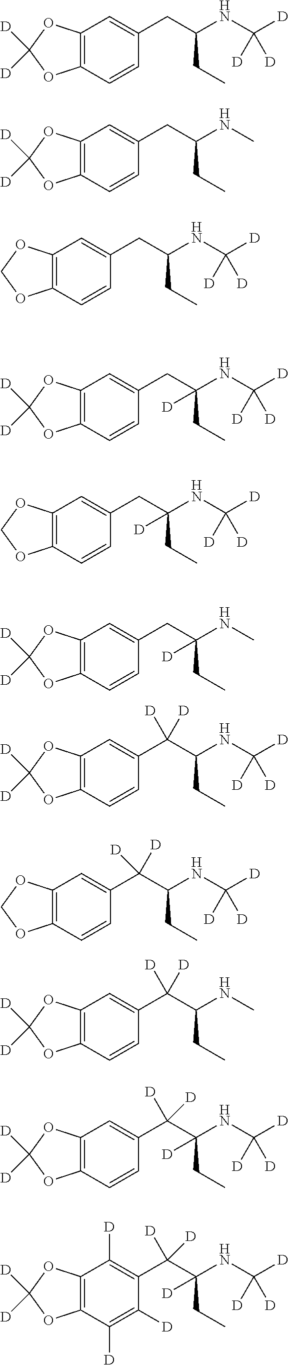

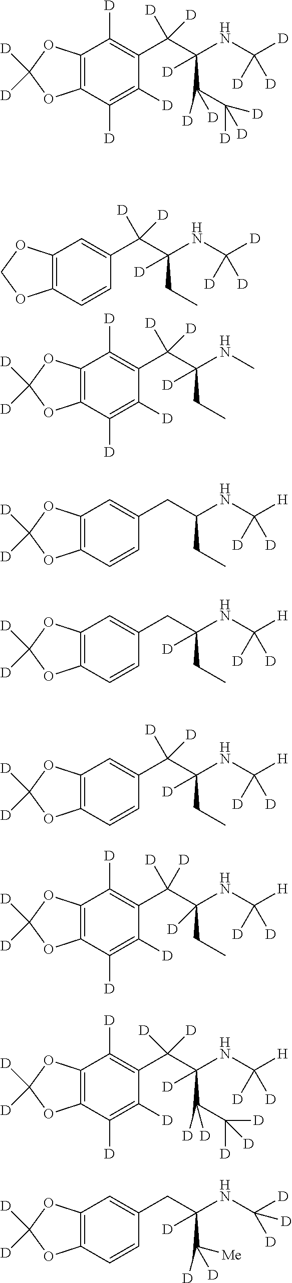

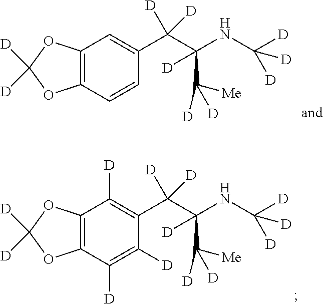 [SMILES: [2H]C([2H])([2H])N[C@@]([2H])(C([2H])([2H])C)C([2H])([2H])c1ccc2c(c1)OC([2H])([2H])O2 ; [2H]c1c([2H])c(C([2H])([2H])[C@@]([2H])(NC([2H])([2H])[2H])C([2H])([2H])C)c([2H])c2c1OC([2H])([2H])O2]

or a formula selected from:

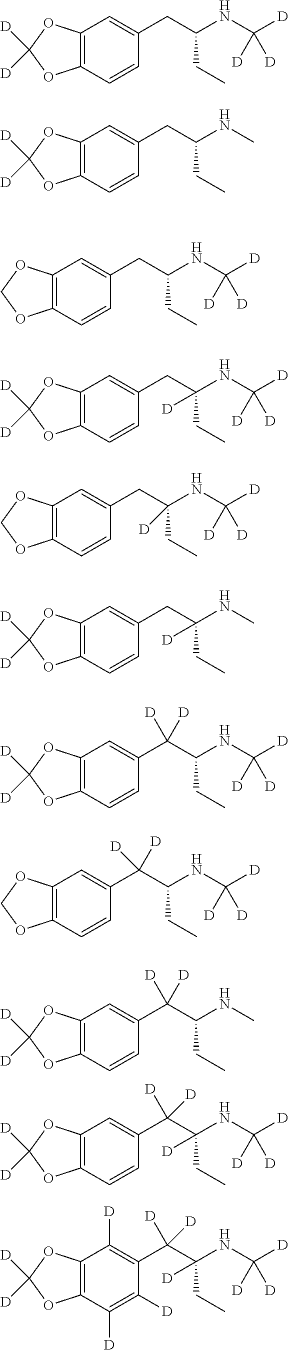

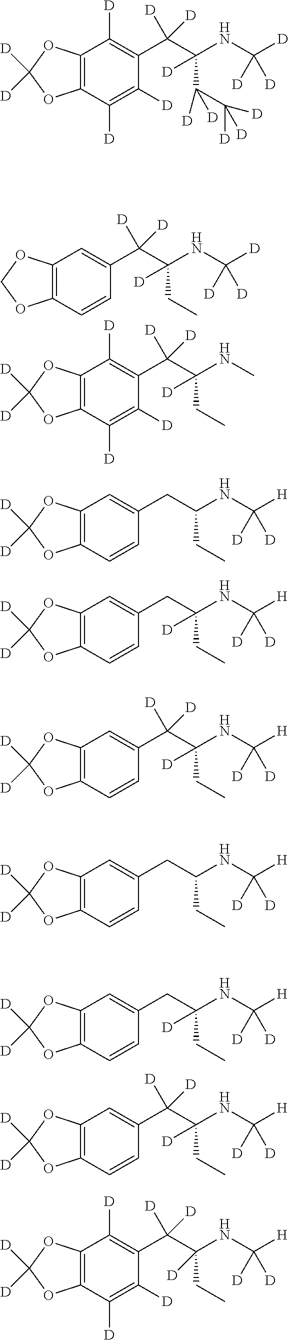

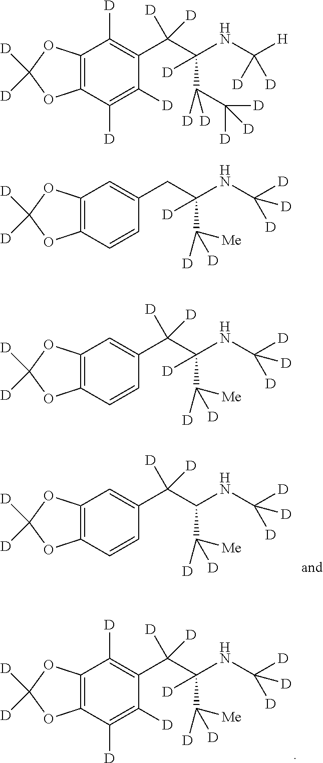

In some embodiments, the present disclosure provides any one of the compounds in Table 1:

TABLE 1

| Cpd | |
|---|---|
| Number | Structure |
| 1 | 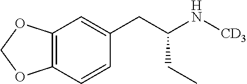 [SMILES: [2H]C([2H])([2H])N[C@H](CC)Cc1ccc2c(c1)OCO2] |
| 2 | 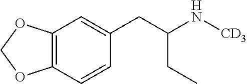 [SMILES: [2H]C([2H])([2H])NC(CC)Cc1ccc2c(c1)OCO2] |
| 3 | 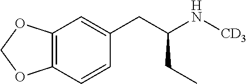 [SMILES: [2H]C([2H])([2H])N[C@@H](CC)Cc1ccc2c(c1)OCO2] |
| 4 | 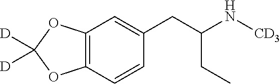 [SMILES: [2H]C([2H])([2H])NC(CC)Cc1ccc2c(c1)OC([2H])([2H])O2] |
| 5 | 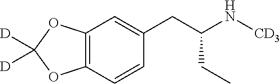 [SMILES: [2H]C([2H])([2H])N[C@H](CC)Cc1ccc2c(c1)OC([2H])([2H])O2] |
| 6 | 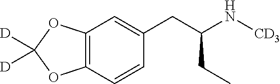 [SMILES: [2H]C([2H])([2H])N[C@@H](CC)Cc1ccc2c(c1)OC([2H])([2H])O2] |
| 7 | 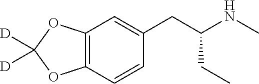 [SMILES: [2H]C1([2H])Oc2ccc(C[C@@H](CC)NC)cc2O1] |
| 8 | 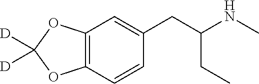 [SMILES: [2H]C1([2H])Oc2ccc(CC(CC)NC)cc2O1] |
| 9 | 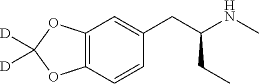 [SMILES: [2H]C1([2H])Oc2ccc(C[C@H](CC)NC)cc2O1] |
| 10 | 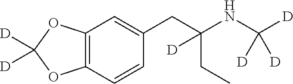 [SMILES: [2H]C([2H])([2H])NC([2H])(CC)Cc1ccc2c(c1)OC([2H])([2H])O2] |
| 11 | 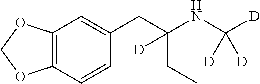 [SMILES: [2H]C([2H])([2H])NC([2H])(CC)Cc1ccc2c(c1)OCO2] |
| 12 | 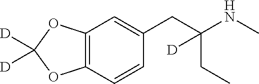 [SMILES: [2H]C(CC)(Cc1ccc2c(c1)OC([2H])([2H])O2)NC] |
| 13 | 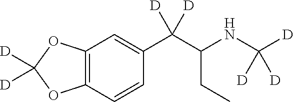 [SMILES: [2H]C([2H])([2H])NC(CC)C([2H])([2H])c1ccc2c(c1)OC([2H])([2H])O2] |
| 14 | 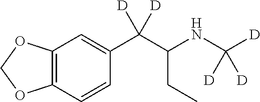 [SMILES: [2H]C([2H])([2H])NC(CC)C([2H])([2H])c1ccc2c(c1)OCO2] |
| 15 | 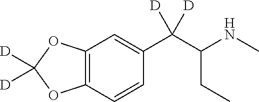 [SMILES: [2H]C1([2H])Oc2ccc(C([2H])([2H])C(CC)NC)cc2O1] |
| 16 | 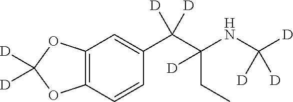 [SMILES: [2H]C([2H])([2H])NC([2H])(CC)C([2H])([2H])c1ccc2c(c1)OC([2H])([2H])O2] |
| 17 | 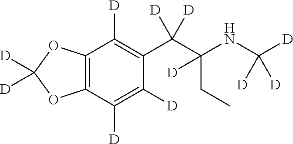 [SMILES: [2H]c1c([2H])c(C([2H])([2H])C([2H])(CC)NC([2H])([2H])[2H])c([2H])c2c1OC([2H])([2H])O2] |
| 18 | 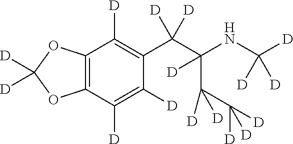 [SMILES: [2H]c1c([2H])c(C([2H])([2H])C([2H])(NC([2H])([2H])[2H])C([2H])([2H])C([2H])([2H])[2H])c([2H])c2c1OC([2H])([2H])O2] |
| 19 | 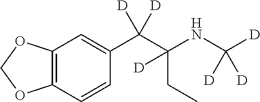 [SMILES: [2H]C([2H])([2H])NC([2H])(CC)C([2H])([2H])c1ccc2c(c1)OCO2] |
| 20 | 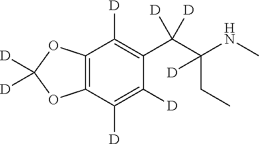 [SMILES: [2H]c1c([2H])c(C([2H])([2H])C([2H])(CC)NC)c([2H])c2c1OC([2H])([2H])O2] |
| 21 | 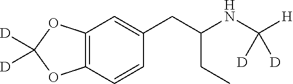 [SMILES: [2H]C([2H])NC(CC)Cc1ccc2c(c1)OC([2H])([2H])O2] |
| 22 | 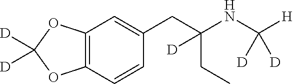 [SMILES: [2H]C([2H])NC([2H])(CC)Cc1ccc2c(c1)OC([2H])([2H])O2] |
| 23 | 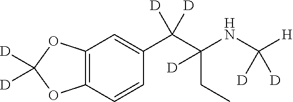 [SMILES: [2H]C([2H])NC([2H])(CC)C([2H])([2H])c1ccc2c(c1)OC([2H])([2H])O2] |
| 24 | 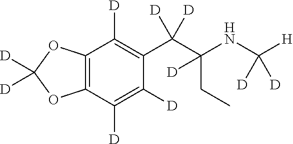 [SMILES: [2H]c1c([2H])c(C([2H])([2H])C([2H])(CC)NC([2H])[2H])c([2H])c2c1OC([2H])([2H])O2] |
| 25 | 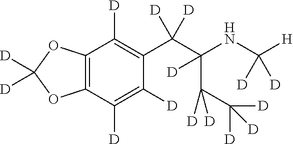 [SMILES: [2H]c1c([2H])c(C([2H])([2H])C([2H])(NC([2H])[2H])C([2H])([2H])C([2H])([2H])[2H])c([2H])c2c1OC([2H])([2H])O2] |
| 26 | 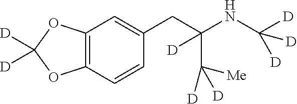 [SMILES: [2H]C([2H])([2H])NC([2H])(Cc1ccc2c(c1)OC([2H])([2H])O2)C([2H])([2H])C] |
| 27 | 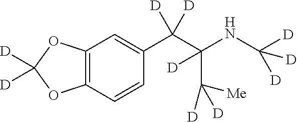 [SMILES: [2H]C([2H])([2H])NC([2H])(C([2H])([2H])C)C([2H])([2H])c1ccc2c(c1)OC([2H])([2H])O2] |
| 28 | 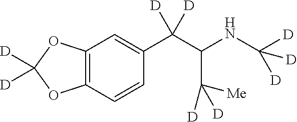 [SMILES: [2H]C([2H])([2H])NC(C([2H])([2H])C)C([2H])([2H])c1ccc2c(c1)OC([2H])([2H])O2] |
| 29 | 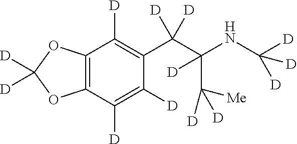 [SMILES: [2H]c1c([2H])c(C([2H])([2H])C([2H])(NC([2H])([2H])[2H])C([2H])([2H])C)c([2H])c2c1OC([2H])([2H])O2] |
| 30 | 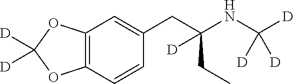 [SMILES: [2H]C([2H])([2H])N[C@@]([2H])(CC)Cc1ccc2c(c1)OC([2H])([2H])O2] |
| 31 | 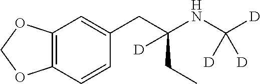 [SMILES: [2H]C([2H])([2H])N[C@@]([2H])(CC)Cc1ccc2c(c1)OCO2] |
| 32 | 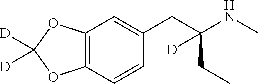 [SMILES: [2H]C1([2H])Oc2ccc(C[C@]([2H])(CC)NC)cc2O1] |
| 33 | 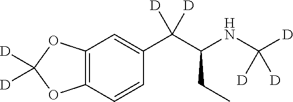 [SMILES: [2H]C([2H])([2H])N[C@@H](CC)C([2H])([2H])c1ccc2c(c1)OC([2H])([2H])O2] |
| 34 | 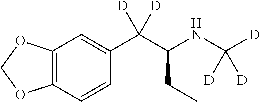 [SMILES: [2H]C([2H])([2H])N[C@@H](CC)C([2H])([2H])c1ccc2c(c1)OCO2] |
| 35 | 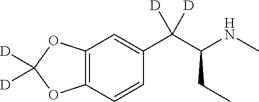 [SMILES: [2H]C1([2H])Oc2ccc(C([2H])([2H])[C@H](CC)NC)cc2O1] |
| 36 | 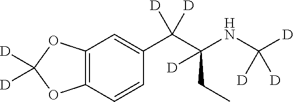 [SMILES: [2H]C([2H])([2H])N[C@@]([2H])(CC)C([2H])([2H])c1ccc2c(c1)OC([2H])([2H])O2] |
| 37 | 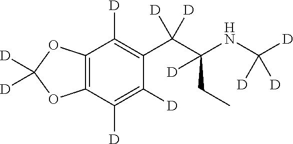 [SMILES: [2H]c1c([2H])c(C([2H])([2H])[C@]([2H])(CC)NC([2H])([2H])[2H])c([2H])c2c1OC([2H])([2H])O2] |
| 38 | 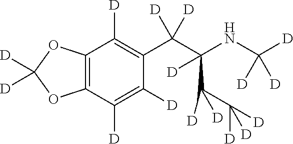 [SMILES: [2H]c1c([2H])c(C([2H])([2H])[C@@]([2H])(NC([2H])([2H])[2H])C([2H])([2H])C([2H])([2H])[2H])c([2H])c2c1OC([2H])([2H])O2] |
| 39 | 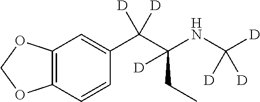 [SMILES: [2H]C([2H])([2H])N[C@@]([2H])(CC)C([2H])([2H])c1ccc2c(c1)OCO2] |
| 40 | 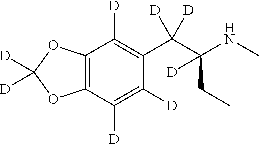 [SMILES: [2H]c1c([2H])c(C([2H])([2H])[C@]([2H])(CC)NC)c([2H])c2c1OC([2H])([2H])O2] |
| 41 | 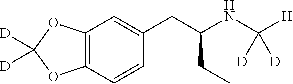 [SMILES: [2H]C([2H])N[C@@H](CC)Cc1ccc2c(c1)OC([2H])([2H])O2] |
| 42 | 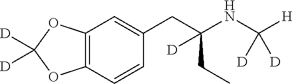 [SMILES: [2H]C([2H])N[C@@]([2H])(CC)Cc1ccc2c(c1)OC([2H])([2H])O2] |
| 43 | 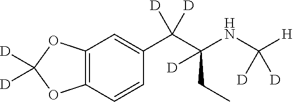 [SMILES: [2H]C([2H])N[C@@]([2H])(CC)C([2H])([2H])c1ccc2c(c1)OC([2H])([2H])O2] |
| 44 | 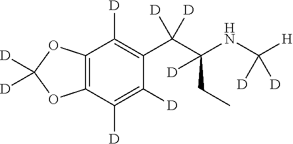 [SMILES: [2H]c1c([2H])c(C([2H])([2H])[C@]([2H])(CC)NC([2H])[2H])c([2H])c2c1OC([2H])([2H])O2] |
| 45 | 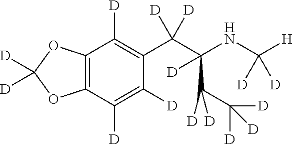 [SMILES: [2H]c1c([2H])c(C([2H])([2H])[C@@]([2H])(NC([2H])[2H])C([2H])([2H])C([2H])([2H])[2H])c([2H])c2c1OC([2H])([2H])O2] |
| 46 | 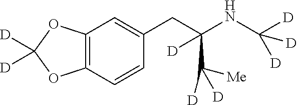 [SMILES: [2H]C([2H])([2H])N[C@]([2H])(Cc1ccc2c(c1)OC([2H])([2H])O2)C([2H])([2H])C] |
| 47 | 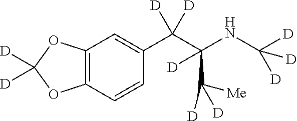 [SMILES: [2H]C([2H])([2H])N[C@@]([2H])(C([2H])([2H])C)C([2H])([2H])c1ccc2c(c1)OC([2H])([2H])O2] |
| 48 | 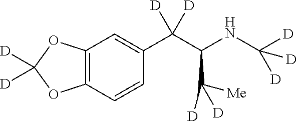 [SMILES: [2H]C([2H])([2H])N[C@@H](C([2H])([2H])C)C([2H])([2H])c1ccc2c(c1)OC([2H])([2H])O2] |
| 49 | 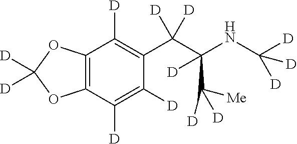 [SMILES: [2H]c1c([2H])c(C([2H])([2H])[C@@]([2H])(NC([2H])([2H])[2H])C([2H])([2H])C)c([2H])c2c1OC([2H])([2H])O2] |
| 50 | 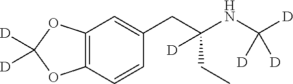 [SMILES: [2H]C([2H])([2H])N[C@]([2H])(CC)Cc1ccc2c(c1)OC([2H])([2H])O2] |
| 51 | 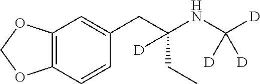 [SMILES: [2H]C([2H])([2H])N[C@]([2H])(CC)Cc1ccc2c(c1)OCO2] |
| 52 | 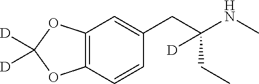 [SMILES: [2H]C1([2H])Oc2ccc(C[C@@]([2H])(CC)NC)cc2O1] |
| 53 | 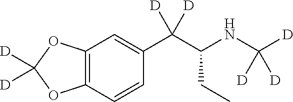 [SMILES: [2H]C([2H])([2H])N[C@H](CC)C([2H])([2H])c1ccc2c(c1)OC([2H])([2H])O2] |
| 54 | 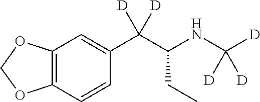 [SMILES: [2H]C([2H])([2H])N[C@H](CC)C([2H])([2H])c1ccc2c(c1)OCO2] |
| 55 | 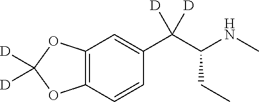 [SMILES: [2H]C1([2H])Oc2ccc(C([2H])([2H])[C@@H](CC)NC)cc2O1] |
| 56 | 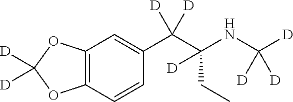 [SMILES: [2H]C([2H])([2H])N[C@]([2H])(CC)C([2H])([2H])c1ccc2c(c1)OC([2H])([2H])O2] |
| 57 | 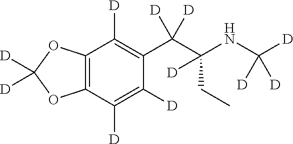 [SMILES: [2H]c1c([2H])c(C([2H])([2H])[C@@]([2H])(CC)NC([2H])([2H])[2H])c([2H])c2c1OC([2H])([2H])O2] |
| 58 | 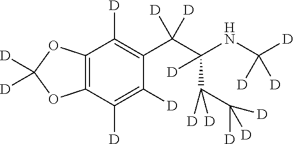 [SMILES: [2H]c1c([2H])c(C([2H])([2H])[C@]([2H])(NC([2H])([2H])[2H])C([2H])([2H])C([2H])([2H])[2H])c([2H])c2c1OC([2H])([2H])O2] |
| 59 | 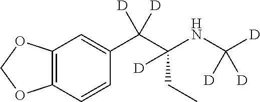 [SMILES: [2H]C([2H])([2H])N[C@]([2H])(CC)C([2H])([2H])c1ccc2c(c1)OCO2] |
| 60 | 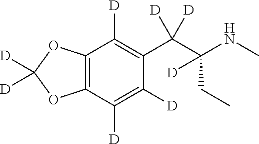 [SMILES: [2H]c1c([2H])c(C([2H])([2H])[C@@]([2H])(CC)NC)c([2H])c2c1OC([2H])([2H])O2] |
| 61 | 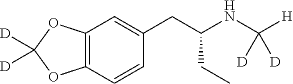 [SMILES: [2H]C([2H])N[C@H](CC)Cc1ccc2c(c1)OC([2H])([2H])O2] |
| 62 | 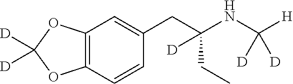 [SMILES: [2H]C([2H])N[C@]([2H])(CC)Cc1ccc2c(c1)OC([2H])([2H])O2] |
| 63 | 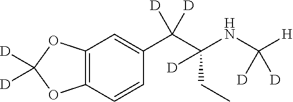 [SMILES: [2H]C([2H])N[C@]([2H])(CC)C([2H])([2H])c1ccc2c(c1)OC([2H])([2H])O2] |
| 64 | 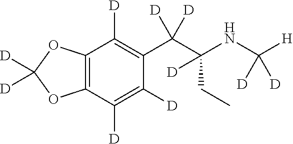 [SMILES: [2H]c1c([2H])c(C([2H])([2H])[C@@]([2H])(CC)NC([2H])[2H])c([2H])c2c1OC([2H])([2H])O2] |
| 65 | 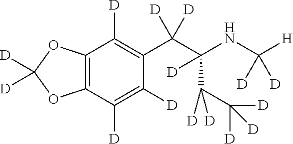 [SMILES: [2H]c1c([2H])c(C([2H])([2H])[C@]([2H])(NC([2H])[2H])C([2H])([2H])C([2H])([2H])[2H])c([2H])c2c1OC([2H])([2H])O2] |
| 66 | 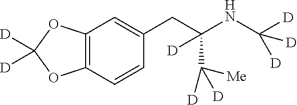 [SMILES: [2H]C([2H])([2H])N[C@@]([2H])(Cc1ccc2c(c1)OC([2H])([2H])O2)C([2H])([2H])C] |
| 67 | 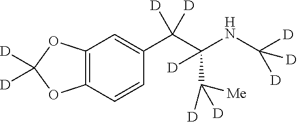 [SMILES: [2H]C([2H])([2H])N[C@]([2H])(C([2H])([2H])C)C([2H])([2H])c1ccc2c(c1)OC([2H])([2H])O2] |
| 68 | 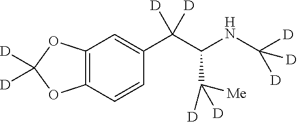 [SMILES: [2H]C([2H])([2H])N[C@H](C([2H])([2H])C)C([2H])([2H])c1ccc2c(c1)OC([2H])([2H])O2] |
| 69 | 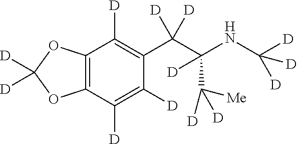 [SMILES: [2H]c1c([2H])c(C([2H])([2H])[C@]([2H])(NC([2H])([2H])[2H])C([2H])([2H])C)c([2H])c2c1OC([2H])([2H])O2] |

The compounds of the present invention can also be in salt forms, such as acid or base salts of the compounds of the present invention. Illustrative examples of pharmaceutically acceptable acid salts are mineral acid (hydrochloric acid, hydrobromic acid, phosphoric acid, and the like) salts, organic acid (fumaric acid, acetic acid, propionic acid, glutamic acid, citric acid, tartaric acid and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The present invention includes all tautomers and stereoisomers of the compounds of the formulas above, including Formulas I and II, and compounds described in Table 1, either in admixture or in pure or substantially pure form. The compounds of the present invention can have asymmetric centers at the carbon atoms, and therefore the compounds of the present invention can exist in diastereomeric or enantiomeric forms or mixtures thereof. All conformational isomers (e.g., cis and trans isomers) and all optical isomers (e.g., enantiomers and diastereomers), racemic, diastereomeric and other mixtures of such isomers.

In addition, all physical forms of the compounds of the Formulas above are intended herein, including the compounds of Formulas I and II and described in Table 1, in the form of solvates, such as hydrates. Moreover, non-crystalline and crystalline forms of the isotopically enriched compounds of Formulas I and II and described in Table 1, including amorphous forms, isomorphs and polymorphs are within the scope of the present invention.

Exemplary compounds according to the present invention are chiral. Such compounds can be prepared as is known to those of skill in the art can be prepared as single enantiomers, or enantiomerically enriched mixtures, or racemic mixtures as contemplated herein; such compounds having more than one stereocenter can also be prepared as diastereomeric, enantiomeric or racemic mixtures as contemplated herein. Furthermore, diastereomer and enantiomer products can be separated by chromatography, fractional crystallization or other methods known to those of skill in the art.

### Pharmaceutical Compositions and Formulations

In some embodiments, the present invention provides a pharmaceutical composition comprising a compound of the present invention, such as a composition comprising a compound of Formulas I and II and in Table 1, illustrated above, and a pharmaceutically acceptable excipient. Such compositions are suitable for administration to a subject, such as a human subject.

The presently disclosed pharmaceutical compositions can be prepared in a wide variety of oral, parenteral and topical dosage forms. Oral preparations include tablets, pills, powder, capsules, liquids, lozenges, cachets, gels, syrups, slurries, suspensions, etc., suitable for ingestion by the patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. The compositions of this invention can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Accordingly, the present invention also provides pharmaceutical compositions including a pharmaceutically acceptable carrier or excipient and the compounds of the present invention.

For preparing pharmaceutical compositions from the compounds disclosed herein, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Mack Publishing Co, Easton Pa. (“Remington's”).

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% to 70% or 10% to 70% of the compounds of the present invention.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen.

If desired, disintegrating or solubilizing agents may be added, such as the cross-linked polyvinyl pyrrolidone, agar, alginic acid, or a salt thereof, such as sodium alginate.

For preparing suppositories, a low melting wax, such as a mixture of fatty acid glycerides or cocoa butter, is first melted and the compounds of the present invention are dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the compounds of the present invention in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of the present invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present invention can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In some embodiments, the pharmaceutical compositions of the present invention can be formulated for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose, any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pFI adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In some embodiments, the formulations of the compositions of the present invention can be delivered by the use of liposomes which fuse with the cellular membrane or are endocytosed, for example, by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

### Administration:

The compositions of the present invention can be administered by any suitable means, including oral, parenteral and topical methods. Transdermal administration methods, by a topical route, can be formulated as applicator sticks, solutions, suspensions, emulsions, gels, creams, ointments, pastes, jellies, paints, powders, and aerosols.

The pharmaceutical preparation is preferably in unit dosage form. In such form the preparation is subdivided into unit doses containing appropriate quantities of the compounds of the present invention. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compound of the present invention can be present in any suitable amount, and can depend on various factors including, but not limited to, weight and age of the subject, state of the disease, and the like as is known to those of ordinary skill in the art. Suitable dosage ranges for the compounds disclosed herein include from about 0.1 mg to about 10,000 mg, or about 1 mg to about 1000 mg, or about 10 mg to about 750 mg, or about 25 mg to about 500 mg, or about 50 mg to about 250 mg. Suitable dosages for the compound of the present invention include about 1 mg, 5, 10, 20, 30, 40, 50, 60, 70, 80, 90, 100, 200, 300, 400, 500, 600, 700, 800, 900 or 1000 mg.

The compounds disclosed herein can be administered at any suitable frequency, interval and duration. For example, the compounds can be administered once an hour, or two, three or more times an hour, once a day, or two, three, or more times per day, or once every 2, 3, 4, 5, 6, or 7 days, so as to provide the preferred dosage level. When the compound of the present invention is administered more than once a day, representative intervals include 5, 10, 15, 20, 30, 45 and 60 minutes, as well as 1, 2, 4, 6, 8, 10, 12, 16, 20, and 24 hours. The compound of the present invention can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

The composition can also contain other compatible therapeutic agents. The compounds described herein can be used in combination with one another, with other active agents known to be useful in modulating a glucocorticoid receptor, or with adjunctive agents that may not be effective alone, but may contribute to the efficacy of the active agent.

The compounds of the present invention can be co-administered with a second active agent. Co-administration includes administering the compound of the present invention and active agent within 0.5, 1, 2, 4, 6, 8, 10, 12, 16, 20, or 24 hours of each other. Co-administration also includes administering the compound of the present invention and active agent simultaneously, approximately simultaneously (e.g., within about 1, 5, 10, 15, 20, or 30 minutes of each other), or sequentially in any order. Moreover, the compound of the present invention and the active agent can each be administered once a day, or two, three, or more times per day so as to provide the preferred dosage level per day.

In some embodiments, co-administration can be accomplished by co-formulation, such as by preparing a single pharmaceutical composition including both the compound of the present invention and a second active agent. In other embodiments, the compound of the present invention and the second active agent can be formulated separately.

The disclosed compounds and the second active agent can be present in the compositions of the present invention in any suitable weight ratio, such as from about 1:100 to about 100:1 (w/w), or about 1:50 to about 50:1, or about 1:25 to about 25:1, or about 1:10 to about 10:1, or about 1:5 to about 5:1 (w/w). The compound of the present invention and the second active agent can be present in any suitable weight ratio, such as about 1:100 (w/w), 1:50, 1:25, 1:10, 1:5, 1:4, 1:3, 1:2, 1:1, 2:1, 3:1, 4:1, 5:1, 10:1, 25:1, 50:1 or 100:1 (w/w). Other dosages and dosage ratios of the compound of the present invention and the active agent are suitable in the compositions and methods disclosed herein.

### Methods of Treatment

The compounds of the present invention, such as a compound of Formulas I and II and described in Table 1, can be used for increasing neuronal plasticity. The compounds of the present invention can also be used to treat any brain disease. The compounds of the present invention can also be used for increasing at least one of translation, transcription or secretion of neurotrophic factors.

In some embodiments, a compound of the present invention, such as a compound of Formulas I and II and in Table 1, is used to treat neurological diseases. In some embodiments, the compounds have, for example, anti-addictive properties, antidepressant properties, anxiolytic properties, or a combination thereof. In some embodiments, the neurological disease is a neuropsychiatric disease. In some embodiments, the neuropsychiatric disease is a mood or anxiety disorder. In some embodiments, the neurological disease is a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder). In some embodiments, the neurological disease is a migraine or cluster headache. In some embodiments, the neurological disease is a neurodegenerative disorder, Alzheimer's disease, or Parkinson's disease. In some embodiments, the neurological disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease or neurological disease is post-traumatic stress disorder (PTSD), addiction (e.g., substance use disorder), schizophrenia, depression, or anxiety. In some embodiments, the neuropsychiatric disease or neurological disease is addiction (e.g., substance use disorder). In some embodiments, the neuropsychiatric disease or neurological disease is depression. In some embodiments, the neuropsychiatric disease or neurological disease is anxiety. In some embodiments, the neuropsychiatric disease or neurological disease is post-traumatic stress disorder (PTSD). In some embodiments, the neurological disease is stroke or traumatic brain injury. In some embodiments, the neuropsychiatric disease or neurological disease is schizophrenia.

In some embodiments, a compound of the present invention is used for increasing neuronal plasticity. In some embodiments, the compounds described herein are used for treating a brain disorder. In some embodiments, the compounds described herein are used for increasing at least one of translation, transcription, or secretion of neurotrophic factors.

In some embodiments, the present invention provides a method of treating a disease, including administering to a subject in need thereof, a therapeutically effective amount of a compound of the present invention, such as a compound of Formulas I and II and in Table 1. In some embodiments, the disease is a musculoskeletal pain disorder including fibromyalgia, muscle pain, joint stiffness, osteoarthritis, rheumatoid arthritis, muscle cramps. In some embodiments, the present invention provides a method of treating a disease of women's reproductive health including premenstrual dysphoric disorder (PMDD), premenstrual syndrome (PMS), post-partum depression, and menopause.

In some embodiments, a single enantiomer of MBDB, e.g., a single enantiomer of isotopically enriched MBDB, e.g., deuterated MBDB, is administered to a subject in order to treat a disorder, e.g., an anxiety disorder or depressive disorder. Anxiety disorders that may be treated with a single enantiomer of MDBD, e.g., deuterated MBDB, include but are not limited to post traumatic stress disorder, generalized anxiety disorder, panic disorder, social anxiety disorder, obsessive-compulsive disorder, separation anxiety disorder, or agoraphobia Depressive disorders that may be treated with a single enantiomer include but are not limited to major depressive disorder, treatment resistant depression, persistent depressive disorder, seasonal effective disorder, premenstrual dysphoric disorder, prolonged grief disorder, bipolar depression, or psychotic depression.

In some embodiments, a single enantiomer of MBDB, e.g., deuterated MBDB, is present in greater than 50% enantiomeric excess (ee) in a composition administered to a subject, e.g., to treat an anxiety disorder and/or depressive disorder. In some embodiments, the single enantiomer of MBDB or an isotopically enriched analog thereof, e.g., S-MBDB and/or deuterated S-MBDB, is present in greater than 60% enantiomeric excess (ee), e.g., the single enantiomer is present in greater than 65% ee, greater than 70% ee, greater than 75% ee, greater than 80% ee, greater than 85% ee, greater than 90% ee, greater than 95% ee, greater than 96% ee, greater than 97% ee, greater than 98% ee, or greater than 99% ee. In some embodiments, the single enantiomer of MBDB is substantially free of the other enantiomer. In some embodiments, a composition comprising S-MBDB that is substantially free of R-MBDB is administered to the subject. In some embodiments, a composition comprising R-MBDB that is substantially free of S-MBDB is administered to the subject.

In some embodiments, administration of a single entantiomer of MBDB, e.g., deuterated MBDB, effectuates fewer and/or less severe side effects, e.g., anxiogenic side effects, in a subject relative to the administration of a racemate of MBDB or the administration or the administration of the alternative enantiomer of MBDB. In some embodiments, administration of S-MBDB, e.g., deuterated S-MBDB, effectuates fewer and/or less severe side effects, e.g., anxiogenic side effects, in a subject relative to administration of racemic MBDB or R-MBDB, e.g., deuterated MBDB or deuterated R-MBDB. In some embodiments, S-MBDB, e.g., deuterated S-MBDB, has a greater therapeutic index than racemic MBDB and R-MBDB, e.g., racemic deuterated MBDB and deuterated R-MBDB, and has a more reduced range of doses that could increase anxiety when compared to racemic MBDB and R-MBDB e.g., racemic deuterated MBDB and deuterated R-MBDB.

In some embodiments, the compounds of the present invention, such as a compound of Formulas I and II and in Table 1, have activity as 5-HT_{2A} modulators. In some embodiments, the compounds of the present invention elicit a biological response by activating the 5-HT_{2A} receptor (e.g., allosteric modulation or modulation of a biological target that activates the 5-HT_{2A} receptor). 5-HT_{2A} agonism has been correlated with the promotion of neural plasticity (Ly et al., 2018). 5-HT_{2A} antagonists abrogate the neuritogenesis and spinogenesis effects of hallucinogenic compounds with 5-HT_{2A} agonist activity, for example, DMT, LSD, and DOI. In some embodiments, the compounds of the present invention are 5-HT_{2A} modulators and promote neural plasticity (e.g., cortical structural plasticity). In some embodiments, the compounds of the present invention are selective 5-HT_{2A} modulators and promote neural plasticity (e.g., cortical structural plasticity). In some embodiments, promotion of neural plasticity includes, for example, increased dendritic spine growth, increased synthesis of synaptic proteins, strengthened synaptic responses, increased dendritic arbor complexity, increased dendritic branch content, increased spinogenesis, increased neuritogenesis, or any combination thereof. In some embodiments, increased neural plasticity includes, for example, increased cortical structural plasticity in the anterior parts of the brain.

In some embodiments, the 5-HT_{2A} modulators (e.g., 5-HT_{2A} agonists) are non-hallucinogenic. In some embodiments, non-hallucinogenic 5-HT_{2A} modulators (e.g., 5-HT_{2A} agonists) are used to treat neurological diseases, which modulators do not elicit dissociative side-effects. In some embodiments, the hallucinogenic potential of the compounds described herein is assessed in vitro. In some embodiments, the hallucinogenic potential assessed in vitro of the compounds described herein is compared to the hallucinogenic potential assessed in vitro of hallucinogenic homologs. In some embodiments, the compounds described herein elicit less hallucinogenic potential in vitro than the hallucinogenic homologs.

In some embodiments, serotonin receptor modulators, such as modulators of serotonin receptor 2A (5-HT_{2A} modulators, e.g., 5-HT_{2A} agonists), are used to treat a brain disorder. The presently disclosed compounds of Formulas I and II and in Table 1 can function as 5-HT_{2A} agonists alone, or in combination with a second therapeutic agent that also is a 5-HT_{2A} modulator. In such cases the second therapeutic agent can be an agonist or an antagonist. In some instances, it may be helpful administer a 5-HT_{2A} antagonist in combination with a compound of the present invention to mitigate undesirable effects of 5-HT_{2A} agonism, such as potential hallucinogenic effects. Serotonin receptor modulators useful as second therapeutic agents for combination therapy as described herein are known to those of skill in the art and include, without limitation, ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methiothepin, methysergide, trazodone, cinitapride, cyproheptadine, brexpiprazole, cariprazine, agomelatine, setoperone, 1-(1-Naphthyl)piperazine, LY-367265, pirenperone, metergoline, deramciclane, amperozide, cinanserin, LY-86057, GSK-215083, cyamemazine, mesulergine, BF-1, LY-215840, sergolexole, spiramide, LY-53857, amesergide, LY-108742, pipamperone, LY-314228, 5-I-R91150, 5-MeO-NBpBrT, 9-Aminomethyl-9,10-dihydroanthracene, niaprazine, SB-215505, SB-204741, SB-206553, SB-242084, LY-272015, SB-243213, SB-200646, RS-102221, zotepine, clozapine, chlorpromazine, sertindole, iloperidone, paliperidone, asenapine, amisulpride, aripiprazole, lurasidone, ziprasidone, lumateperone, perospirone, mosapramine, AMDA (9-Aminomethyl-9,10-dihydroanthracene), methiothepin, an extended-release form of olanzapine (e.g., ZYPREXA RELPREVV), an extended-release form of quetiapine, an extended-release form of risperidone (e.g., Risperdal Consta), an extended-release form of paliperidone (e.g., Invega Sustenna and Invega Trinza), an extended-release form of fluphenazine decanoate including Prolixin Decanoate, an extended-release form of aripiprazole lauroxil including Aristada, and an extended-release form of aripiprazole including Abilify Maintena, or a pharmaceutically acceptable salt, solvate, metabolite, deuterated analog, derivative, prodrug, or combinations thereof. In some embodiments, the serotonin receptor modulator used as a second therapeutic is pimavanserin or a pharmaceutically acceptable salt, solvate, metabolite, derivative, or prodrug thereof. In some embodiments, the serotonin receptor modulator is administered prior to a compound disclosed herein, such as about three or about one hours prior to administration of a compound according to Formulas I and II or Table 1. In some embodiments, the serotonin receptor modulator is administered at most about one hour prior to the presently disclosed compound. Thus, in some embodiments of combination therapy with the presently disclosed compounds, the second therapeutic agent is a serotonin receptor modulator. In some embodiments the second therapeutic agent serotonin receptor modulator is provided at a dose of from about 10 mg to about 350 mg. In some embodiments, the serotonin receptor modulator is provided at a dose of from about 20 mg to about 200 mg. In some embodiments, the serotonin receptor modulator is provided at a dose of from about 10 mg to about 100 mg. In certain such embodiments, the compound of the present invention is provided at a dose of from about 10 mg to about 100 mg, or from about 20 to about 200 mg, or from about 15 to about 300 mg, and the serotonin receptor modulator is provided at a dose of about 10 mg to about 100 mg. In certain such embodiments, the compound of the present invention is provided at a dose of from about 10 mg to about 500 mg, or from about 150 mg to about 250 mg, or about 180 mg, or about 210 mg, or about 250 mg, and the serotonin receptor modulator is provided at a dose of about 10 mg to about 100 mg.

In some embodiments, non-hallucinogenic 5-HT2_{A} modulators (e.g., 5-HT2_{A} agonists) are used to treat neurological diseases. In some embodiments, the neurological diseases comprise decreased neural plasticity, decreased cortical structural plasticity, decreased 5-HT_{2A} receptor content, decreased dendritic arbor complexity, loss of dendritic spines, decreased dendritic branch content, decreased spinogenesis, decreased neuritogenesis, retraction of neurites, or any combination thereof.

In some embodiments, non-hallucinogenic 5-HT_{2A} modulators (e.g., 5-HT_{2A} agonists) are used for increasing neuronal plasticity. In some embodiments, non-hallucinogenic 5-HT_{2A} modulators (e.g., 5-HT_{2A} agonists) are used for treating a brain disorder. In some embodiments, non-hallucinogenic 5-HT_{2A} modulators (e.g., 5-FIT_{2A} agonists) are used for increasing at least one of translation, transcription, or secretion of neurotrophic factors.

In some embodiments the presently disclosed compounds of Formulas I and II and described in Table 1 are given to patients in a low dose that is lower than would produce noticeable psychedelic effects but high enough to provide a therapeutic benefit. In some embodiments, this dose range is between 200 ug (micrograms) and 2 mg.

### Methods of Increasing Neuronal Plasticity

Neuronal plasticity refers to the ability of the brain to change structure and/or function throughout a subject's life. New neurons can be produced and integrated into the central nervous system throughout the subject's life. Increasing neuronal plasticity includes, but is not limited to, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing neuronal plasticity comprises promoting neuronal growth, promoting neuritogenesis, promoting synaptogenesis, promoting dendritogenesis, increasing dendritic arbor complexity, and increasing dendritic spine density.

In some embodiments, increasing neuronal plasticity by treating a subject with a compound disclosed herein, e.g., a compound of Formulas I and II or Table 1 can treat neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, the present invention provides methods for increasing neuronal plasticity, comprising contacting a neuronal cell with a compound of the present invention, such as a compound of Formulas I and II or Table 1. In some embodiments, increasing neuronal plasticity improves a brain disorder described herein.

In some embodiments, a compound of the present invention is used to increase neuronal plasticity. In some embodiments, the compounds used to increase neuronal plasticity have, for example, anti-addictive properties, antidepressant properties, anxiolytic properties, or a combination thereof. In some embodiments, decreased neuronal plasticity is associated with a neuropsychiatric disease. In some embodiments, the neuropsychiatric disease is a mood or anxiety disorder. In some embodiments, the neuropsychiatric disease includes, for example, migraine, cluster headache, post-traumatic stress disorder (PTSD), schizophrenia, anxiety, depression, and addiction (e.g., substance abuse disorder). In some embodiments, brain disorders include, for example, migraines, addiction (e.g., substance use disorder), depression, and anxiety.

In some embodiments, the experiment or assay to determine increased neuronal plasticity of any compound of the present invention is a phenotypic assay, a dendritogenesis assay, a spinogenesis assay, a synaptogenesis assay, a Sholl analysis, a concentration-response experiment, a 5-HT_{2A} agonist assay, a 5-HT_{2A} antagonist assay, a 5-HT_{2A} binding assay, or a 5-HT_{2A} blocking experiment (e.g., ketanserin blocking experiments). In some embodiments, the experiment or assay to determine the hallucinogenic potential of any compound of the present invention is a mouse head-twitch response (HTR) assay.

In some embodiments, the present invention provides a method for increasing neuronal plasticity, comprising contacting a neuronal cell with a compound of Formulas I and II or Table 1.

### Methods of Treating a Brain Disorder

In some embodiments, the present invention provides a method of treating a disease, including administering to a subject in need thereof, a therapeutically effective amount of a compound of the present invention, such as a compound of Formulas I and II or Table 1. In some embodiments, the disease is a musculoskeletal pain disorder including fibromyalgia, muscle pain, joint stiffness, osteoarthritis, rheumatoid arthritis, muscle cramps. In some embodiments, the present invention provides a method of treating a disease of women's reproductive health including premenstrual dysphoric disorder (PMDD), premenstrual syndrome (PMS), post-partum depression, and menopause. In some embodiments, the present invention provides a method of treating a brain disorder, including administering to a subject in need thereof, a therapeutically effective amount of a compound of the present invention. In some embodiments, the present invention provides a method of treating a brain disorder with combination therapy, including administering to a subject in need thereof, a therapeutically effective amount of a compound of the present invention and at least one additional therapeutic agent.

In some embodiments, 5-HT_{2A} modulators (e.g., 5-HT_{2A} agonists) are used to treat a brain disorder. In some embodiments, the brain disorders comprise decreased neural plasticity, decreased cortical structural plasticity, decreased 5-HT_{2A} receptor content, decreased dendritic arbor complexity, loss of dendritic spines, decreased dendritic branch content, decreased spinogenesis, decreased neuritogenesis, retraction of neurites, or any combination thereof.

In some embodiments, a compound of the present invention, such as a compound of Formulas I and II or Table 1, is used to treat brain disorders. In some embodiments, the compounds have, for example, anti-addictive properties, antidepressant properties, anxiolytic properties, or a combination thereof. In some embodiments, the brain disorder is a neuropsychiatric disease. In some embodiments, the neuropsychiatric disease is a mood or anxiety disorder. In some embodiments, brain disorders include, for example, migraine, cluster headache, post-traumatic stress disorder (PTSD), anxiety, depression, panic disorder, suicidality, schizophrenia, and addiction (e.g., substance abuse disorder). In some embodiments, brain disorders include, for example, migraines, addiction (e.g., substance use disorder), depression, and anxiety.

In some embodiments, the present invention provides a method of treating a brain disorder, comprising administering to a subject in need thereof a therapeutically effective amount of a compound disclosed herein, such as a compound of Formulas I and II or Table 1.

In some embodiments, the brain disorder is a neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, the brain disorder is a neurodegenerative disorder, Alzheimer's, or Parkinson's disease. In some embodiments, the brain disorder is a psychological disorder, depression, addiction, anxiety, or a post-traumatic stress disorder. In some embodiments, the brain disorder is depression. In some embodiments, the brain disorder is addiction. In some embodiments, the brain disorder is treatment resistant depression, suicidal ideation, major depressive disorder, bipolar disorder, schizophrenia, stroke, traumatic brain injury or substance use disorder. In some embodiments, the brain disorder is treatment resistant depression, suicidal ideation, major depressive disorder, bipolar disorder, schizophrenia, or substance use disorder. In some embodiments, the brain disorder is stroke or traumatic brain injury. In some embodiments, the brain disorder is treatment resistant depression, suicidal ideation, major depressive disorder, bipolar disorder, or substance use disorder. In some embodiments, the brain disorder is schizophrenia. In some embodiments, the brain disorder is alcohol use disorder.

In some embodiments, the method further comprises administering one or more additional therapeutic agent that is lithium, olanzapine (Zyprexa), quetiapine (Seroquel), risperidone (Risperdal), ariprazole (Abilify), ziprasidone (Geodon), clozapine (Clozaril), divalproex sodium (Depakote), lamotrigine (Lamictal), valproic acid (Depakene), carbamazepine (Equetro), topiramate (Topamax), levomilnacipran (Fetzima), duloxetine (Cymbalta, Yentreve), venlafaxine (Effexor), citalopram (Celexa), fluvoxamine (Luvox), escitalopram (Lexapro), fluoxetine (Prozac), paroxetine (Paxil), sertraline (Zoloft), clomipramine (Anafranil), amitriptyline (Elavil), desipramine (Norpramin), imipramine (Tofranil), nortriptyline (Pamelor), phenelzine (Nardil), tranylcypromine (Parnate), diazepam (Valium), alprazolam (Xanax), or clonazepam (Klonopin).

In certain embodiments of the method for treating a brain disorder disclosed herein with a compound described herein, such as a compound according to Formulas I and II or described in Table 1, a second therapeutic agent that is an empathogenic agent is administered. Examples of suitable empathogenic agents for use in combination with a compound according to Formulas I and II or in Table 1 are selected from the phenethylamines, such as 3,4-methylene-dioxymethamphetamine (MDMA) and analogs thereof. Other suitable empathogenic agents for use in combination with the presently disclosed compounds include, without limitation,

N-Allyl-3,4-methylenedioxy-amphetamine (MDAL)
N-Butyl-3,4-methylenedioxyamphetamine (MDBU)
N-Benzyl-3,4-methylenedioxyamphetamine (MDBZ)
N-Cyclopropylmethyl-3,4-methylenedioxyamphetamine (MDCPM)
N,N-Dimethyl-3,4-methylenedioxyamphetamine (MDDM)
N-Ethyl-3,4-methylenedioxyamphetamine (MDE; MDEA)
N-(2-Hydroxyethyl)-3,4-methylenedioxy amphetamine (MDHOET)
N-Isopropyl-3,4-methylenedioxyamphetamine (MDIP)
N-Methyl-3,4-ethylenedioxyamphetamine (MDMC)
N-Methoxy-3,4-methylenedioxyamphetamine (MDMEO)
N-(2-Methoxyethyl)-3,4-methylenedioxyamphetamine (MDMEOET)
alpha,alpha,N-Trimethyl-3,4-methylenedioxyphenethylamine (MDMP;

### 3,4-Methylenedioxy-N-methylphentermine)

N-Hydroxy-3,4-methylenedioxyamphetamine (MDOH)

### 3,4-Methylenedioxyphenethylamine (MDPEA)

alpha,alpha-Dimethyl-3,4-methylenedioxyphenethylamine (MDPH; 3,4-methylenedioxyphentermine)
N-Propargyl-3,4-methylenedioxyamphetamine (MDPL)
Methylenedioxy-2-aminoindane (MDAI)
3,4-methylenedioxy-N-methyl-α-ethylphenylethylamine

### 3,4-Methylenedioxyamphetamine MDA

Methylone (also known as “3,4-methylenedioxy-N-methylcathinone)
Ethylone, also known as 3,4-methylenedioxy-N-ethylcathinone
GHB or Gamma Hydroxybutyrate or sodium oxybate
N-Propyl-3,4-methylenedioxyamphetamine (MDPR), and the like.

In some embodiments, the compounds of the present invention are used in combination with the standard of care therapy for a neurological disease described herein. Non-limiting examples of the standard of care therapies, may include, for example, lithium, olanzapine, quetiapine, risperidone, ariprazole, ziprasidone, clozapine, divalproex sodium, lamotrigine, valproic acid, carbamazepine, topiramate, levomilnacipran, duloxetine, venlafaxine, citalopram, fluvoxamine, escitalopram, fluoxetine, paroxetine, sertraline, clomipramine, amitriptyline, desipramine, imipramine, nortriptyline, phenelzine, tranylcypromine, diazepam, alprazolam, clonazepam, or any combination thereof. Nonlimiting examples of standard of care therapy for depression are sertraline, fluoxetine, escitalopram, venlafaxine, or aripiprazole. Non-limiting examples of standard of care therapy for depression are citralopram, escitalopram, fluoxetine, paroxetine, diazepam, or sertraline. Additional examples of standard of care therapeutics are known to those of ordinary skill in the art.

Methods of Increasing Translation, Transcription, and/or Secretion of Neurotrophic Factors

Neurotrophic factors refers to a family of soluble peptides or proteins which support the survival, growth, and differentiation of developing and mature neurons. Increasing at least one of translation, transcription, or secretion of neurotrophic factors can be useful for, but not limited to, increasing neuronal plasticity, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing at least one of translation, transcription, or secretion of neurotrophic factors can increasing neuronal plasticity. In some embodiments, increasing at least one of translation, transcription, or secretion of neurotrophic factors can promoting neuronal growth, promoting neuritogenesis, promoting synaptogenesis, promoting dendritogenesis, increasing dendritic arbor complexity, and/or increasing dendritic spine density.

In some embodiments, 5-HT_{2A} modulators (e.g., 5-HT_{2A} agonists) are used to increase at least one of translation, transcription, or secretion of neurotrophic factors. In some embodiments, a compound of the present invention, such as a compound of Formulas I and II or Table 1, is used to increase at least one of translation, transcription, or secretion of neurotrophic factors. In some embodiments, increasing at least one of translation, transcription or secretion of neurotrophic factors treats a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder).

In some embodiments, the experiment or assay used to determine increase translation of neurotrophic factors includes ELISA, western blot, immunofluorescence assays, proteomic experiments, and mass spectrometry. In some embodiments, the experiment or assay used to determine increase transcription of neurotrophic factors includes gene expression assays, PCR, and microarrays. In some embodiments, the experiment or assay used to determine increase secretion of neurotrophic factors includes ELISA, western blot, immunofluorescence assays, proteomic experiments, and mass spectrometry.

In some embodiments, the present invention provides a method for increasing at least one of translation, transcription or secretion of neurotrophic factors, comprising contacting a neuronal cell with a compound disclosed herein, such as a compound of Formulas I and II or Table 1.

### EXAMPLES

Exemplary compounds disclosed herein are prepared from the isotopically enriched building blocks analogous to those used to synthesize the unenriched compounds. Where exemplary compounds may be presented as a salt, a skilled artisan would understand the present disclosure encompasses the free base forms as well.

### General Conditions:

Mass spectra were run on LC-MS systems using electrospray ionization. These were run using a Waters Acquity Classic UPLC with PDA and SQ mass detection or a Waters Acquity H-Class UPLC with PDA and QDA mass detection. [M+H]⁺ refers to mono-isotopic molecular weights. NMR spectra were run on either a Bruker Ultrashield 400 MHz or 500 MHz NMR spectrometer. Spectra were recorded at 298 K, unless otherwise stated, and were referenced using the solvent peak.

The following examples are intended to illustrate the invention and are not to be construed as being limitations thereon. Temperatures are given in degrees centigrade. If not mentioned otherwise, all evaporations are performed in vacuo, preferably between about 15 mm Hg and 100 mm Hg (=20-133 mbar). The structure of final products, intermediates and starting materials is confirmed by standard analytical methods, e.g., MS and NMR. Abbreviations used are those conventional in the art. If not defined, the terms have their generally accepted meanings.

### Abbreviation

app apparent
br broad
CDCl₃ chloroform-d
D₂O deuterium oxide
d doublet
d deuterium
dd doublet of doublets
DCM dichloromethane
DIPEA diisopropylethylamine
DMA dimethylacetamide
DMAP 4-dimethylaminopyridine

### DMF N,N-dimethylformamide

DMSO dimethyl sulfoxide
Et₂O diethyl ether
EtOAc ethyl acetate
HCl hydrochloric acid
h hextet; sextet
HPLC high pressure liquid chromatography
LC-MS liquid chromatography and mass spectrometry
MeOH methanol
MeCN acetonitrile
MS mass spectrometry
m multiplet
min(s) minute(s)
mL milliliter(s)
L microliter(s)
m/z mass to charge ratio
p pentet
q quartet
N₂ nitrogen
NaHCO₃ sodium hydrogen carbonate
NaOH sodium hydroxide
Na₂SO₄ sodium sulfate
NH₄Cl ammonium chloride
NMP N-methyl-2-pyrrolidone
NMR nuclear magnetic resonance
Rt retention time
s singlet
t triplet
Ts tosyl
tert tertiary
THF tetrahydrofuran

Referring to the examples that follow, compounds of the preferred embodiments were synthesized using the methods described herein, or other methods, which are known in the art. The various starting materials, intermediates, and compounds of the preferred embodiments may be isolated and purified, where appropriate, using conventional techniques such as precipitation, filtration, crystallization, evaporation, distillation, and chromatography. Salts may be prepared from compounds by known salt-forming procedures. Unless otherwise stated, all starting materials are obtained from commercial suppliers and used without further purification. If not indicated otherwise, the analytical IPLC conditions are as follows:

### Instrument: LC-MS-1:

### Method 2A

Column: Acquity UPLC BEH C18 2.1×50 mm 1.7 μm
Column Temp: 50° C.
Flow rate: 0.8 mL/min.
Eluents: A: H₂O, 0.1% formic acid, B: MeCN
Gradient: 0.0-1.8 min 2-98% B, 1.8-2.1 min 98% B, 2.1-2.5 98% A.

### Method 2B

Column: Acquity UPLC BEH C18 2.1×50 mm 1.7 μm
Column Temp: 50° C.
Flow rate: 0.8 mL/min.
Eluents: A: H₂O, 0.1% ammonia B: MeCN
Gradient: 0.0-1.8 min 2-98% B, 1.8-2.1 min 98% B, 2.1-2.5 98% A.

### Instrument: LC-MS-2:

### Method 2A

Column: Acquity UPLC BEH C18 2.1×50 mm 1.7 μm
Column Temp: 50° C.
Flow rate: 0.8 mL/min.
Eluents: A: H₂O, B: MeCN, C: 50% H₂O/50% MeCN+2.0% formic acid
Gradient: 0.0-1.7 mins 0-95% B, 5% C; 1.7-2.1 mins 95% B, 5% C 2.1-2.5 mins 95% A, 5% C.

### Method 2B

Column: Acquity UPLC BEH C18 2.1×50 mm 1.7 μm
Column Temp: 50° C.
Flow rate: 0.8 mL/min.
Eluents: A: H₂O, B: MeCN, C: 50% H₂O/50% MeCN+2.0% ammonia (aq.)
Gradient: 0.0-1.7 mins 0-95% B, 5% D; 1.7-2.1 mins 95% B, 5% D 2.1-2.5 mins 95% A, 5% D.

### Example 1: 4-Bromo-1,2-dideuteriooxy-benzene

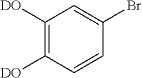

A solution of 4-bromobenzene-1,2-diol (5.37 g, 28.4 mmol) in THF (15 mL) and D₂O (15 mL) was stirred at rt under N₂ overnight. The mixture was concentrated in vacuo. Methanol-d4 (15 mL) and D₂O (15 mL) were added to the residue and the mixture was then stirred at rt under N₂ for 3 days. The mixture was concentrated in vacuo to leave 4-bromo-1,2-dideuteriooxy-benzene (5.20 g, 96% yield) as a solid. Spectroscopic data of the title compound showed 85% deuterium incorporation. The intermediate was used in the next step without further purification. ¹H NMR (400 MHz, CDCl₃) δ 7.02 (d, J=2.3 Hz, 1H), 6.93 (dd, J=8.4, 2.3 Hz, 1H), 6.74 (d, J=8.4 Hz, 1H), 5.32 (br. s, 0.15H), 5.17 (br. s, 0.15H).

### Example 2: 5-Bromo-2,2-dideuterio-1,3-benzodioxole

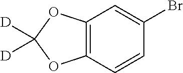 [SMILES: [2H]C1([2H])Oc2ccc(Br)cc2O1]

A solution of 4-bromo-1,2-dideuteriooxy-benzene (4.68 g, 24.5 mmol) in DCM-d2 (6.39 g, 73.5 mmol, 4.73 mL) and NMP (4.5 mL) was added dropwise over 5 min to a stirred suspension of cesium carbonate (15.97 g, 49.0 mmol) in NMP (9 mL) and D₂O (1.47 g, 73.5 mmol, 1.33 mL) at rt under N₂. The mixture was stirred at 110° C. for 2 h. The mixture was cooled to rt and then filtered. Water (50 mL) and EtOAc (50 mL) were added to the filtrate. The separated aqueous phase was extracted with EtOAc (2×25 mL) and the combined organic fractions were then washed with brine (50 mL), dried over Na₂SO₄ and concentrated in vacuo. The residue was purified by chromatography on silica, eluting with 10% EtOAc in petroleum ether, to leave 5-bromo-2,2-dideuterio-1,3-benzodioxole (3.10 g, 62% yield) as an oil. Spectroscopic data of the title compound showed 99% deuterium incorporation. LC-MS (LCMS2: Method 2A): Rt 1.56 mins; MS m/z not observed; ¹H NMR (400 MHz, CDCl₃) δ 6.98-6.91 (m, 2H), 6.69 (d, J=8.2 Hz, 1H).

### Example 3: (2S)-1-(2,2-Dideuterio-1,3-benzodioxol-5-yl)butan-2-ol

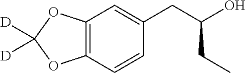 [SMILES: [2H]C1([2H])Oc2ccc(C[C@@H](O)CC)cc2O1]

A solution of n-butyllithium (11.8 mmol, 7.39 mL, 1.6 M in hexanes) was added dropwise over 10 min to a stirred solution of 5-bromo-2,2-dideuterio-1,3-benzodioxole (2.00 g, 9.85 mmol) in THF (15 mL) at −78° C. under N₂. The mixture was stirred at −78° C. for 15 min and then (S)-(−)-butylene oxide (426 mg, 5.91 mmol, 509 μL) was added dropwise over 5 min. The mixture was stirred at −78° C. for 10 min and then boron trifluoride diethyl etherate (1.40 g, 9.85 mmol, 1.24 mL) was added dropwise over 5 min. The mixture was stirred at −78° C. for 30 min and then a saturated aqueous NH₄Cl solution (50 mL) was added, followed by Et₂O (50 mL). The separated aqueous phase was extracted with Et₂O (100 mL). The combined organic fractions were washed with saturated aqueous NH₄Cl solution (40 mL), dried over Na₂SO₄ and concentrated in vacuo. The residue was purified by chromatography on silica, eluting with 0-20% acetone in petroleum ether, to leave (2S)-1-(2,2-dideuterio-1,3-benzodioxol-5-yl)butan-2-ol (774 mg, 40% yield) as an oil. LC-MS (LCMS2: Method 2A): Rt 1.33 mins; MS m/z 179.1=[M-OH]+; ¹H NMR (400 MHz, CDCl₃) δ 6.76 (d, J=7.9 Hz, 1H), 6.72 (d, J=1.7 Hz, 1H), 6.66 (dd, J=7.9, 1.7 Hz, 1H), 3.73-3.65 (m, 1H), 2.76 (dd, J=13.7, 4.2 Hz, 1H), 2.55 (dd, J=13.7, 8.4 Hz, 1H), 1.62-1.44 (m, 3H), 0.99 (t, J=7.4 Hz, 3H).

The compounds of the following tabulated Examples (Table Ex3) were prepared analogously to Example 3 from the appropriate bromide.

TABLE Ex3

| Structure and Name | Retention Time, [M + H]⁺, ¹H NMR |
|---|---|
| 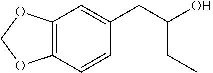 | LC-MS (LCMS2: Method 2A): Rt 1.34 mins; MS m/z 177.1 = [M − OH]⁺ ¹H NMR (400 MHz, CDCl₃) δ 6.75 (d, J = 7.9 Hz, 1H), 6.71 (d, J = 1.7 Hz, 1H), 6.66 (dd, J = 7.9, 1.7 Hz, 1H), 5.93 (s, 2H), 3.72- |
| 1-(1,3-Benzodioxol-5-yl)butan-2-ol | 3.64 (m, 1H), 2.75 (dd, J = 13.7, 4.2 Hz, |
| | 1H), 2.55 (dd, J = 13.7, 8.4 Hz, 1H), 1.60- |
| | 1.44 (m, 3H), 0.99 (t, J = 7.5 Hz, 3H). |
| 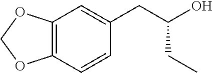 | LC-MS (LCMS2: Method 2A): Rt 1.33 mins; MS m/z 177.0 = [M − OH]⁺ ¹H NMR (400 MHz, CDCl₃) δ 6.76 (d, J = 7.9 Hz, 1H), 6.72 (s, 1H), 6.66 (d, J = 7.9 Hz, 1H), 5.93 (s, 2H), 3.73-3.64 (m, 1H), |
| (2R)-1-(1,3-Benzodioxol-5-yl)butan-2-ol | 2.76 (dd, J = 13.7, 4.2 Hz, 1H), 2.56 (dd, J = |
| | 13.7, 8.4 Hz, 1H), 1.61-1.45 (m, 3H), 0.99 |
| | (t, J = 7.4 Hz, 3H). |
| 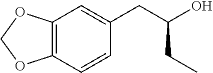 | LC-MS (LCMS2: Method 2B): Rt 1.37 mins; MS m/z 177.2 = [M − OH]⁺ ¹H NMR (400 MHz, CDCl₃) δ 6.76 (d, J = 7.9 Hz, 1H), 6.72 (d, J = 1.7 Hz, 1H), 6.66 (dd, J = 7.9, 1.7 Hz, 1H), 5.93 (s, 2H), 3.73- |
| (2S)-1-(1,3-Benzodioxol-5-yl)butan-2-ol | 3.64 (m, 1H), 2.75 (dd, J = 13.7, 4.2 Hz, |
| | 1H), 2.55 (dd, J = 13.7, 8.4 Hz, 1H), 1.61- |
| | 1.44 (m, 3H), 0.99 (t, J = 7.5 Hz, 3H). |
| 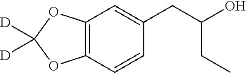 [SMILES: [2H]C1([2H])Oc2ccc(CC(O)CC)cc2O1] | LC-MS (LCMS2: Method 2A): Rt 1.34 mins; MS m/z 179.1 = [M − OH]⁺ ¹H NMR (400 MHz, CDCl₃) δ 6.76 (d, J = 7.9 Hz, 1H), 6.72 (d, J = 1.6 Hz, 1H), 6.66 (dd, J = 7.9, 1.6 Hz, 1H), 3.73-3.65 (m, |
| 1-(2,2-Dideuterio-1,3-benzodioxol-5- | 1H), 2.76 (dd, J = 13.7, 4.2 Hz, 1H), 2.56 |
| yl)butan-2-ol | (dd, J = 13.7, 8.4 Hz, 1H), 1.62-1.44 (m, |
| | 3H), 0.99 (t, J = 7.4 Hz, 3H). |
| 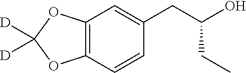 [SMILES: [2H]C1([2H])Oc2ccc(C[C@H](O)CC)cc2O1] | LC-MS (LCMS2: Method 2A): Rt 1.59 mins; MS m/z 179.1 = [M − OH]⁺ ¹H NMR (400 MHz, CDCl₃) δ 6.76 (d, J = 7.9 Hz, 1H), 6.72 (d, J = 1.6 Hz, 1H), 6.66 (dd, J = 7.9, 1.6 Hz, 1H), 3.73-3.65 (m, |
| (2R)-1-(2,2-Dideuterio-1,3-benzodioxol-5- | 1H), 2.76 (dd, J = 13.7, 4.2 Hz, 1H), 2.56 |
| yl)butan-2-ol | (dd, J = 13.7, 8.4 Hz, 1H), 1.61-1.44 (m, |
| | 3H), 0.99 (t, J = 7.5 Hz, 3H). |

### Example 4: [(1S)-1-(1,3-Benzodioxol-5-ylmethyl)propyl] 4-methylbenzenesulfonate

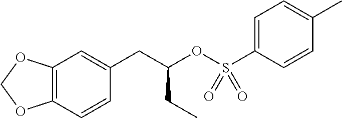 [SMILES: CC[C@@H](Cc1ccc2c(c1)OCO2)OS(=O)(=O)c1ccc(C)cc1]

p-Toluenesulfonyl chloride (1.65 g, 8.65 mmol) was added in several portions over 20 min to a stirred solution of (2S)-1-(1,3-benzodioxol-5-yl)butan-2-ol (1.12 g, 5.77 mmol) in pyridine (10 mL) at 0° C. under N₂. The mixture was stirred at 0° C. for 1 h and then warmed to rt overnight. p-Toluenesulfonyl chloride (275 mg, 1.44 mmol) was added in one portion to the mixture at 0° C. The mixture was stirred at 0° C. for 1 h, warmed to rt and then stirred at this temperature for 90 min. The mixture was poured on to ice (50 g) and then chloroform (50 mL) was added. The separated organic phase was washed with 1 M aqueous HCl (2×100 mL), dried over Na₂SO₄ and then concentrated in vacuo. Heptane (50 mL) was added to the residue and the mixture was stirred at 50° C. for 10 min. The mixture was cooled to −20° C. for 2 hours. The liquid was decanted away from the oil. This process was repeated. The final solid was dried under vacuum to leave [(1S)-1-(1,3-benzodioxol-5-ylmethyl)propyl] 4-methylbenzenesulfonate (1.62 g, 81% yield) as a solid. The product was used without further purification. LC-MS (LCMS2: Method 2A): Rt 1.80 mins; MS m/z 177.1=[M-OTs]+; ¹H NMR (400 MHz, CDCl₃) δ 7.69-7.62 (m, 2H), 7.26-7.23 (m, 2H), 6.63 (d, J=8.3 Hz, 1H), 6.53-6.48 (m, 2H), 5.91 (s, 2H), 4.61-4.53 (in, 1H), 2.81 (dd, J=14.1, 6.4 Hz, 1H), 2.75 (dd, J=14.1, 6.4 Hz, 1H), 2.43 (s, 3H), 1.73-1.56 (m, 2H), 0.86 (t, J=7.4 Hz, 3H).

The compounds of the following tabulated Examples (Table Ex4) were prepared analogously to Example 4 from the appropriate secondary alcohol.

TABLE Ex4

| Structure and Name | Retention Time, [M + H]⁺, ¹H NMR |
|---|---|
| 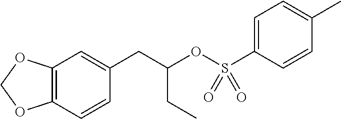 | LC-MS (LCMS2: Method 2A): Rt 1.79 mins; MS m/z 177.1 = [M − OTs]⁺ ¹H NMR (400 MHz, CDCl₃) δ 7.70-7.62 (m, 2H), 7.26-7.22 (m, 2H), 6.63 (d, J = 8.3 Hz, 1H), 6.54-6.48 (m, 2H), 5.91 (s, 2H), 4.60-4.53 (m, 1H), 2.81 (dd, J = 14.1, 6.4 Hz, 1H), 2.75 (dd, J = 14.1, 6.4 Hz, 1H), 2.42 (s, 3H), 1.71-1.56 (m, 2H), 0.86 (t, |
| 1-(1,3-Benzodioxol-5- | J = 7.4 Hz, 3H). |
| ylmethyl)propyl 4- | |
| methylbenzenesulfonate | |
| 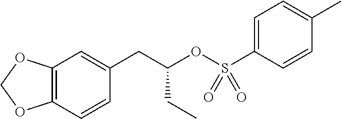 | LC-MS (LCMS2: Method 2A): Rt 1.80 mins; MS m/z 177.1 = [M − OTs]⁺ ¹H NMR (400 MHz, CDCl₃) δ 7.68-7.64 (m, 2H), 7.26-7.23 (m, 2H), 6.63 (d, J = 8.3 Hz, 1H), 6.53-6.48 (m, 2H), 5.91 (s, 2H), 4.60-4.53 (m, 1H), 2.81 (dd, J = 14.1, 6.5 Hz, 1H), 2.75 (dd, J = 14.1, 6.5 Hz, 1H), 2.43 (s, 3H), 1.71-1.58 (m, 2H), 0.86 (t, |
| [(1R)-1-(1,3-Benzodioxol-5- | J = 7.4 Hz, 3H). |
| ylmethyl)propyl] 4- | |
| methylbenzenesulfonate | |
| 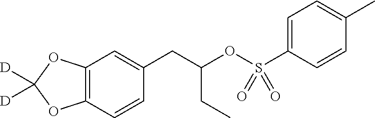 | LC-MS (LCMS2: Method 2A): Rt 1.80 mins; MS m/z 179.1 = [M − OTs]⁺ ¹H NMR (400 MHz, CDCl₃) δ 7.68-7.63 (m, 2H), 7.25-7.23 (m, 2H), 6.63 (d, J = 8.3 Hz, 1H), 6.53-6.48 (m, 2H), 4.60- 4.54 (m, 1H), 2.81 (dd, J = 14.0, 6.4 Hz, 1H), 2.75 (dd, J = 14.0, 6.4 Hz, 1H), 2.43 (s, 3H), 1.70-1.58 (m, 2H), 0.86 (t, J = 7.4 |
| 1-[(2,2-Dideuterio-1,3-benzodioxol- | Hz, 3H). |
| 5-yl)methyl]propyl 4- | |
| methylbenzenesulfonate | |
| 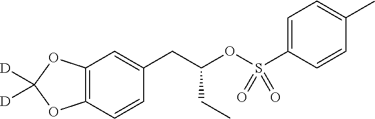 | LC-MS (LCMS2: Method 2A): Rt 1.66 mins; MS m/z 179.0 = [M − OTs]⁺ ¹H NMR (400 MHz, CDCl₃) δ 7.68-7.63 (m, 2H), 7.26-7.22 (m, 2H), 6.63 (d, J = 8.3 Hz, 1H), 6.54-6.47 (m, 2H), 4.60- 4.53 (m, 1H), 2.82 (dd, J = 14.1, 6.4 Hz, 1H), 2.75 (dd, J = 14.1, 6.4 Hz, 1H), 2.43 (s, 3H), 1.72-1.57 (m, 2H), 0.86 (t, J = 7.4 |
| [(1R)-1-[(2,2-Dideuterio-1,3- | Hz, 3H). |
| benzodioxol-5-yl)methyl]propyl] 4- | |
| methylbenzenesulfonate | |
| 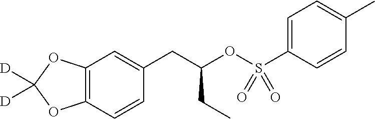 | LC-MS (LCMS2: Method 2A): Rt 1.79 mins; MS m/z 179.1 = [M − OTs]⁺ ¹H NMR (400 MHz, CDCl₃) δ 7.68-7.64 (m, 2H), 7.26-7.22 (m, 2H), 6.63 (d, J = 8.3 Hz, 1H), 6.53-6.47 (m, 2H), 4.60- 4.53 (m, 1H), 2.81 (dd, J = 14.1, 6.5 Hz, 1H), 2.75 (dd, J = 14.1, 6.5 Hz, 1H), 2.43 (s, 3H), 1.71-1.57 (m, 2H), 0.86 (t, J = 7.4 |
| [(1S)-1-[(2,2-Dideuterio-1,3- | Hz, 3H). |
| benzodioxol-5-yl)methyl]propyl] 4- | |
| methylbenzenesulfonate | |

### Example 5: (2R)-1-(1,3-Benzodioxol-5-yl)-N-(trideuteriomethyl)butan-2-amine hydrochloride (compound 1)

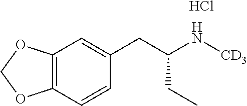 [SMILES: Cl ; [2H]C([2H])([2H])N[C@H](CC)Cc1ccc2c(c1)OCO2]

Sodium hydroxide (294 mg, 7.35 mmol) was added in one portion to a stirred suspension of [(1S)-1-(1,3-benzodioxol-5-ylmethyl)propyl] 4-methylbenzenesulfonate (320 mg, 0.92 mmol) and methyl-d3-amine hydrochloride (518 mg, 7.35 mmol) in THF (5 mL) at rt under N₂. The mixture was stirred at rt for 1 h and then at 90° C. for 3 days. The mixture was cooled to rt and then the liquid was decanted away from the solid. THF (5 mL) was added to the solid and then the liquid was decanted again. This process was repeated a further two times. The combined organic fractions were concentrated in vacuo. EtOAc (20 mL) and 2 M aqueous sodium hydroxide (20 mL) were added to the residue. The separated aqueous phase was extracted with EtOAc (2×20 mL) and the combined organic fractions were then dried over Na₂SO₄ and concentrated in vacuo. The residue was dissolved in MeOH (5 mL) and then the solution was purified using an SCX-2 cartridge, eluting with MeOH (50 mL), MeCN (50 mL), MeOH (50 mL) and then 2 M ammonia in MeOH (2×25 mL), to leave an oil. The residue was dissolved in Et₂O (6 mL) and 1,4-dioxane (2 mL) and then 4 M HCl in dioxane (0.5 mL) was added. The precipitated solid was collected by filtration, washed with Et₂O (4×2 mL), and then the solid was dried under vacuum at 50° C. to leave (2R)-1-(1,3-benzodioxol-5-yl)-N-(trideuteriomethyl)butan-2-amine hydrochloride (131 mg, 57% yield) as a solid. LC-MS (LCMS2: Method 2A): Rt 0.97 mins; MS m/z 211.2=[M+H]⁺; LC-MS (LCMS2: Method 2B): Rt 1.43 mins; MS m/z 211.2=[M+H]⁺; ¹H NMR (400 MHz, D₂O) δ 6.90 (d, J=8.0 Hz, 1H), 6.87 (s, 1H), 6.82 (d, J=8.0 Hz, 1H), 5.98 (s, 2H), 3.40 (app. p, J=7.1 Hz, 1H), 2.99 (dd, J=14.5, 6.8 Hz, 1H), 2.91 (dd, J=14.5, 7.4 Hz, 1H), 1.79-1.65 (m, 2H), 1.00 (t, J=7.5 Hz, 3H). NH and HCl not observed; ²H NMR (61 MHz, H₂O) δ 2.62 (s, 3D).

The compounds of the following tabulated Examples (Table Ex5) were prepared analogously to Example 5 from the appropriate tosylate.

TABLE Ex5

| Cpd | | |
|---|---|---|
| Number | Structure and Name | Retention Time, [M + H]⁺, ¹H NMR |
| 2 | 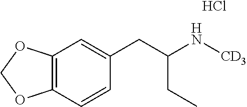 [SMILES: Cl ; [2H]C([2H])([2H])NC(CC)Cc1ccc2c(c1)OCO2] | LC-MS (LCMS2: Method 2A): Rt 0.93 mins; MS m/z 211.1 = [M + H]⁺ LC-MS (LCMS2: Method 2B): Rt 1.34 mins; MS m/z 211.1 = [M + H]⁺ ¹H NMR (400 MHz, D₂O) δ 6.90 (d, J = 7.9 Hz, 1H), 6.87 (s, 1H), 6.82 (d, J = 7.9 Hz, 1H), 5.98 (s, 2H), 3.40 (app. p, J = 7.2 Hz, |
| | 1-(1,3-Benzodioxol-5-yl)-N- | 1H), 2.98 (dd, J = 14.5, 6.9 Hz, 1H), 2.91 |
| | (trideuteriomethyl)butan-2- | (dd, J = 14.5, 7.5 Hz, 1H), 1.79-1.65 (m, |
| | amine hydrochloride | 2H), 1.00 (t, J = 7.5 Hz, 3H). NH and HCl |
| | | not observed. |
| | | ²H NMR (61 MHz, H₂O) δ 2.61 (s, 3D). |
| 3 | 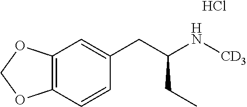 [SMILES: Cl ; [2H]C([2H])([2H])N[C@@H](CC)Cc1ccc2c(c1)OCO2] | LC-MS (LCMS2: Method 2A): Rt 0.91 mins; MS m/z 211.1 = [M + H]⁺ LC-MS (LCMS2: Method 2B): Rt 1.33 mins; MS m/z 211.1 = [M + H]⁺ ¹H NMR (400 MHz, CDCl₃) δ 6.90 (d, J = 8.0 Hz, 1H), 6.88 (d, J = 1.8 Hz, 1H), 6.82 (dd, J = 8.0, 1.8 Hz, 1H), 5.98 (s, 2H), 3.44- |
| | (2S)-1-(1,3-Benzodioxol-5-yl)- | 3.37 (m, 1H), 2.99 (dd, J = 14.5, 6.9 Hz, |
| | N-(trideuteriomethyl)butan-2- | 1H), 2.91 (dd, J = 14.5, 7.5 Hz, 1H), 1.79- |
| | amine hydrochloride | 1.66 (m, 2H), 1.00 (t, J = 7.5 Hz, 3H). NH |
| | | and HCl not observed. |
| | | ²H NMR (61 MHz, H₂O) δ 2.61 (s, 3D). |
| 4 | 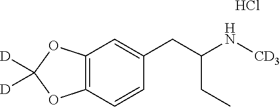 [SMILES: Cl ; [2H]C([2H])([2H])NC(CC)Cc1ccc2c(c1)OC([2H])([2H])O2] | LC-MS (LCMS2: Method 2A): Rt 0.90 mins; MS m/z 213.1 = [M + H]⁺ LC-MS (LCMS2: Method 2B): Rt 1.41 mins; MS m/z 213.1 = [M + H]⁺ ¹H NMR (400 MHz, D₂O) δ 6.90 (d, J = 7.9 Hz, 1H), 6.87 (s, 1H), 6.82 (d, J = 7.9 Hz, 1H), 3.40 (app. p, J = 7.2 Hz, 1H), 2.99 (dd, |
| | 1-(2,2-Dideuterio-1,3- | J = 14.5, 6.9 Hz, 1H), 2.91 (dd, J = 14.5, 7.5 |
| | benzodioxol-5-yl)-N- | Hz, 1H), 1.80-1.64 (m, 2H), 1.00 (t, J = |
| | (trideuteriomethyl)butan-2- | 7.5 Hz, 3H). NH and HCl not observed. |
| | amine hydrochloride | ²H NMR (61 MHz, H₂O) δ 5.92 (s, 2D), |
| | | 2.61 (s, 3D). |
| 5 | 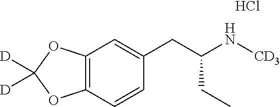 [SMILES: Cl ; [2H]C([2H])([2H])N[C@H](CC)Cc1ccc2c(c1)OC([2H])([2H])O2] | LC-MS (LCMS2: Method 2A): Rt 0.91 mins; MS m/z 213.2 = [M + H]⁺ LC-MS (LCMS2: Method 2B): Rt 1.41 mins; MS m/z 213.2 = [M + H]⁺ ¹H NMR (400 MHz, D₂O) δ 6.90 (d, J = 8.0 Hz, 1H), 6.87 (d, J = 1.7 Hz, 1H), 6.81 (dd, J = 8.0, 1.7 Hz, 1H), 3.40 (app. p, J = 7.2 |
| | (2R)-1-(2,2-Dideuterio-1,3- | Hz, 1H), 2.99 (dd, J = 14.5, 6.9 Hz, 1H), |
| | benzodioxol-5-yl)-N- | 2.91 (dd, J = 14.5, 7.5 Hz, 1H), 1.79-1.65 |
| | (trideuteriomethyl)butan-2- | (m, 2H), 1.00 (t, J = 7.5 Hz, 3H). NH and |
| | amine hydrochloride | HCl not observed. |
| | | ²H NMR (61 MHz, H₂O) δ 5.92 (s, 2D), |
| | | 2.62 (s, 3D). |
| 6 | 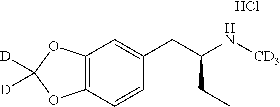 [SMILES: Cl ; [2H]C([2H])([2H])N[C@@H](CC)Cc1ccc2c(c1)OC([2H])([2H])O2] | LC-MS (LCMS2: Method 2A): Rt 0.96 mins; MS m/z 213.2 = [M + H]⁺ LC-MS (LCMS2: Method 2B): Rt 1.37 mins; MS m/z 213.2 = [M + H]⁺ ¹H NMR (400 MHz, D₂O) δ 6.90 (d, J = 8.0 Hz, 1H), 6.87 (s, 1H), 6.82 (d, J = 8.0 Hz, 1H), 3.40 (app. p, J = 7.2 Hz, 1H), 2.99 (dd, |
| | (2S)-1-(2,2-Dideuterio-1,3- | J = 14.5, 6.9 Hz, 1H), 2.91 (dd, J = 14.5, 7.5 |
| | benzodioxol-5-yl)-N- | Hz, 1H), 1.79-1.64 (m, 2H), 1.00 (t, J = |
| | (trideuteriomethyl)butan-2- | 7.5 Hz, 3H). NH and HCl not observed. |
| | amine hydrochloride | ²H NMR (61 MHz, H₂O) δ 5.92 (s, 2D), |
| | | 2.61 (s, 3D). |

### Example 6: (2R)-1-(2,2-Dideuterio-1,3-benzodioxol-5-yl)-N-methyl-butan-2-amine hydrochloride (compound 7)

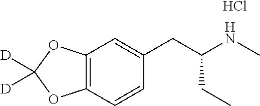 [SMILES: Cl ; [2H]C1([2H])Oc2ccc(C[C@@H](CC)NC)cc2O1]

A solution of [(1S′)-1-[(2,2-dideuterio-1,3-benzodioxol-5-yl)methyl]propyl] 4-methylbenzenesulfonate (308 mg, 0.88 mmol) in THF (1 mL) and a solution of methylamine (16.0 mmol, 8.00 mL, 2.0 M in THF) was stirred in a sealed vessel at 90° C. under N₂ for 3 days. The mixture was cooled to rt and then concentrated in vacuo. EtOAc (20 mL) and 2 M aqueous NaOH (20 mL) were added to the residue. The separated aqueous phase was extracted with EtOAc (2×20 mL) and the combined organic fractions were then dried over Na₂SO₄ and concentrated in vacuo. The residue was dissolved in MeOH (5 mL) and then the solution was purified using an SCX-2 cartridge, eluting with MeOH (50 mL), MeCN (50 mL), MeOH (50 mL) and then 2 M ammonia in MeOH (2×25 mL), to leave an oil. The residue was dissolved in Et₂O (6 mL) and 1,4-dioxane (2 mL) and then 4 M HCl in dioxane (0.5 mL) was added. The precipitated solid was collected by filtration, washed with Et₂O (3×5 mL), and then the solid was dried under vacuum at 50° C. to leave (2R)-1-(2,2-dideuterio-1,3-benzodioxol-5-yl)-N-methyl-butan-2-amine hydrochloride (157 mg, 72% yield) as a solid. LC-MS (LCMS2: Method 2A): Rt 0.91 mins; MS m/z 210.2=[M+H]⁺; LC-MS (LCMS2: Method 2B): Rt 1.41 mins; MS m/z 210.2=[M+H]⁺; ¹H NMR (400 MHz, D₂O) δ 6.90 (d, J=8.0 Hz, 1H), 6.87 (d, J=1.7 Hz, 1H), 6.81 (dd, J=8.0, 1.7 Hz, 1H), 3.41 (app. p, J=7.2 Hz, 1H), 2.99 (dd, J=14.5, 6.9 Hz, 1H), 2.91 (dd, J=14.5, 7.5 Hz, 1H), 2.69 (s, 3H), 1.79-1.65 (m, 2H), 1.00 (t, J=7.5 Hz, 3H). NH and HCl not observed; ²H NMR (61 MHz, H₂O) δ 5.92 (s, 2D).

The compounds of the following tabulated Examples (Table Ex6) were prepared analogously to Example 6 from the appropriate tosylate.

TABLE Ex6

| Cpd | | |
|---|---|---|
| Number | Structure and Name | Retention Time, [M + H]⁺, ¹H NMR |
| 8 | 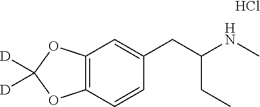 [SMILES: Cl ; [2H]C1([2H])Oc2ccc(CC(CC)NC)cc2O1] | LC-MS (LCMS2: Method 2A): Rt 0.93 mins; MS m/z 210.2 = [M + H]⁺ LC-MS (LCMS2: Method 2B): Rt 1.42 mins; MS m/z 210.2 = [M + H]⁺ ¹H NMR (400 MHz, D₂O) δ 6.90 (d, J = 8.0 Hz, 1H), 6.87 (d, J = 1.7 Hz, 1H), 6.82 (dd, J = 8.0, 1.7 Hz, 1H), 3.40 (app. p, J = 7.2 |
| | 1-(2,2-Dideuterio-1,3- | Hz, 1H), 2.99 (dd, J = 14.5, 6.9 Hz, 1H), |
| | benzodioxol-5-yl)-N-methyl- | 2.91 (dd, J = 14.5, 7.5 Hz, 1H), 2.68 (s, 3H), |
| | butan-2-amine hydrochloride | 1.79-1.66 (m, 2H), 1.00 (t, J = 7.5 Hz, |
| | | 3H). NH and HCl not observed. |
| | | ²H NMR (61 MHz, H₂O) δ 5.93 (s, 2D). |
| 9 | 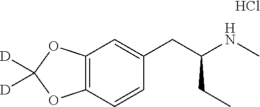 [SMILES: Cl ; [2H]C1([2H])Oc2ccc(C[C@H](CC)NC)cc2O1] | LC-MS (LCMS2: Method 2A): Rt 0.98 mins; MS m/z 210.2 = [M + H]⁺ ¹H NMR (400 MHz, D₂O) δ 6.90 (d, J = 8.0 Hz, 1H), 6.87 (d, J = 1.7 Hz, 1H), 6.82 (dd, J = 8.0, 1.7 Hz, 1H), 3.40 (app. p, J = 7.2 Hz, 1H), 2.99 (dd, J = 14.5, 6.9 Hz, 1H), 2.91 (dd, J = 14.5, 7.5 Hz, 1H), 2.68 (s, 3H), |
| | (2S)-1-(2,2-Dideuterio-1,3- | 1.79-1.65 (m, 2H), 1.00 (t, J = 7.5 Hz, |
| | benzodioxol-5-yl)-N-methyl- | 3H). NH and HCl not observed. |
| | butan-2-amine hydrochloride | ²H NMR (61 MHz, H₂O) δ 5.93 (s, 2D). |

### Example 7: Evaluation of Metabolic Stability in Human Liver Microsomes

Microsomal Assay: Human liver microsomes (20 mg/mL) were obtained. β-nicotinamide adenine dinucleotide phosphate, reduced form (NADPH), magnesium chloride (MgCl₂), and dimethyl sulfoxide (DMSO) were purchased from Sigma-Aldrich.

Determination of Metabolic Stability: 7.5 mM stock solutions of test compounds of the above structural formula (e.g., of an embodiment or aspect of embodiment thereof described herein), or pharmaceutically acceptable salt thereof, are prepared in DMSO. The 7.5 mM stock solutions were diluted to 12.5-50 μM in acetonitrile (ACN). The 20 mg/mL human liver microsomes were diluted to 0.625 mg/mL in 0.1 M potassium phosphate buffer, pH 7.4, containing 3 mM MgCl₂. The diluted microsomes were added to wells of a 96-well deep-well polypropylene plate in triplicate. A 10 μL aliquot of the 12.5-50 μM test compound was added to the microsomes and the mixture is pre-warmed for 10 minutes. Reactions were initiated by addition of pre-warmed NADPH solution. The final reaction volume is 0.5 mL and contains 4.0 mg/mL human liver microsomes, 0.25 μM test compound, and 2 mM NADPH in 0.1 M potassium phosphate buffer, pH 7.4, and 3 mM MgCl₂. The reaction mixtures were incubated at 37° C., and 50 μL aliquots were removed at 0, 5, 10, 20, and 30 minutes and added to shallow-well 96-well plates which contain 50 μL of ice-cold ACN (acetonitrile) with internal standard to stop the reactions. The plates were stored at 4° C. for 20 minutes after which 100 μL of water was added to the wells of the plate before centrifugation to pellet precipitated proteins. Supernatants were transferred to another 96-well plate and analyzed for amounts of parent remaining by LC-MS/MS using an Applied Bio-systems API 4000 mass spectrometer. The same procedure was followed for the non-enriched counterpart of the compound and the positive control, 7-ethoxycoumarin (1 μM). Testing was done in triplicate.

Data analysis: The in vitro T/s for test compounds were calculated from the slopes of the linear regression of % parent remaining (In) vs incubation time relationship.

in vitro T_{1/2}=0.693/k

k=−[slope of linear regression of % parent remaining (In) vs incubation time]
The apparent intrinsic clearance was calculated using the following equation:

CLᵢₙₜ (mL/min/kg)=(0.693/in vitro T)(Incubation Volume/mg of microsomes)(45 mg microsomes/gram of liver) (20 gm of liver/kg b.w.)

Data analysis was performed using Microsoft Excel Software.

In these experiments, values equal to or more than a 15% increase in half-life are considered to be a significant difference if the apparent intrinsic clearance ratio (isotopically enriched compound/MBDB) is >1.15 or <0.85, then there is considered to be significant differentiation.

TABLE EX7
Metabolic stability in human liver microsomes
of representative deuterated compounds

| | | CLᵢₙₜ | | |
|---|---|---|---|---|
| Compound | | (μL/min/ | SE | t_{1/2} |
| number | Compound name | mg protein) | CLᵢₙₜ | (min) |
| — | rac-MBDB HCl salt | 11.9 | 5.33 | 116 |
| | (reference compound) | | | |
| — | R-MBDB HCl salt | 11.7 | 4.30 | 119 |
| | (reference compound) | | | |
| — | S-MBDB HCl salt | 8.06 | 3.48 | 172 |
| | (reference compound) | | | |
| 8 | 1-(2,2-Dideuterio-1,3- | 17.2 | 3.85 | 80.6 |
| | benzodioxol-5-yl)-N- | | | |
| | methyl-butan-2-amine | | | |
| | hydrochloride | | | |
| | [rac-MBDB-D2 HCl] | | | |
| 7 | (2R)-1-(2,2-Dideuterio- | 36.7 | 3.62 | 37.7 |
| | 1,3-benzodioxol-5-yl)-N- | | | |
| | methyl-butan-2-amine | | | |
| | hydrochloride | | | |
| | [R-MBDB-D2 HCl] | | | |
| 9 | (2S)-1-(2,2-Dideuterio- | 26.7 | 5.35 | 52.0 |
| | 1,3-benzodioxol-5-yl)-N- | | | |
| | methyl-butan-2-amine | | | |
| | hydrochloride | | | |
| | [S-MBDB-D2 HCl] | | | |
| 4 | 1-(2,2-Dideuterio-1,3- | 33.0 | 2.02 | 42.0 |
| | benzodioxol-5-yl) -N- | | | |
| | (trideuteriomethyl)butan- | | | |
| | 2-amine hydrochloride | | | |
| | [rac-MBDB-D5 HCl] | | | |
| 5 | (2R)-1-(2,2-Dideuterio- | 57.1 | 8.40 | 24.3 |
| | 1,3-benzodioxol-5-yl)-N- | | | |
| | (trideuteriomethyl)butan- | | | |
| | 2-amine hydrochloride | | | |
| | [R-MBDB-D5 HCl] * | | | |
| 6 | (25)-1-(2,2-Dideuterio- | 25.4 | 5.57 | 54.6 |
| | 1,3-benzodioxol-5-yl)-N- | | | |
| | (trideuteriomethyl)butan- | | | |
| | 2-amine hydrochloride | | | |
| | [S-MBDB-D5 HCl] | | | |
| 2 | 1-(1,3-Benzodioxol-5-yl)- | 12.4 | 4.73 | 112 |
| | N- | | | |
| | (trideuteriomethyl)butan- | | | |
| | 2-amine hydrochloride | | | |
| | [rac-MBDB-ND3 HCl] | | | |
| 1 | (2R)-1-(1,3-Benzodioxol- | 14.3 | 5.47 | 96.8 |
| | 5-yl)-N- | | | |
| | (trideuteriomethyl)butan- | | | |
| | 2-amine hydrochloride | | | |
| | [R-MBDB-ND3 HCl] | | | |
| 3 | (2S)-1-(1,3-Benzodioxol- | 8.04 | 3.13 | 173 |
| | 5-yl)-N- | | | |
| | (trideuteriomethyl)butan- | | | |
| | 2-amine hydrochloride | | | |
| | [S-MBDB-ND3 HCl] | | | |
| — | dextromethorphan** | 53.4 | 3.42 | 26.0 |
| | (positive control) | | | |
| — | verapamil*** | 322.3 | 14.75 | 4.33 |
| | (positive control) | | | |

* 45-minute time point excluded due to it plateauing. This is common and is standard procedure to exclude those points. If included, it does not give an accurate clearance and would make the clearance appear slower.
**Average of n = 3 experiments.
***Average of n = 3 experiments; 30- and 45-minute time points excluded because verapamil is metabolized very fast and is completely gone by 30 min. Thus, the 30 min and 45 min timepoints are not able to be measured.

Based on the results in Table Ex7, Compounds 1-(2,2-Dideuterio-1,3-benzodioxol-5-yl)-N-methyl-butan-2-amine hydrochloride [rac-MBDB-D2 HCl] (compound 8) and 1-(2,2-Dideuterio-1,3-benzodioxol-5-yl)-N-(trideuteriomethyl)butan-2-amine hydrochloride [rac-MBDB-D5 HCl] (compound 4) exhibit significant differences in half-life and intrinsic clearance compared to rac-MBDB HCl salt (reference compound). Compound 1-(1,3-Benzodioxol-5-yl)-N-(trideuteriomethyl)butan-2-amine hydrochloride [rac-MBDB-ND3 HCl] (compound 2) also demonstrates differences in half-life and intrinsic clearance compared to rac-MBDB HCl (reference compound).

Compounds (2R)-1-(2,2-Dideuterio-1,3-benzodioxol-5-yl)-N-methyl-butan-2-amine hydrochloride [R-MBDB-D2 HCl] (compound 7), (2R)-1-(2,2-Dideuterio-1,3-benzodioxol-5-yl)-N-(trideuteriomethyl)butan-2-amine hydrochloride [R-MBDB-D5 HCl] (compound 5) and (2R)-1-(1,3-Benzodioxol-5-yl)-N-(trideuteriomethyl)butan-2-amine hydrochloride [R-MBDB-ND3 HCl] (compound 1) exhibit significant differences in half-life and intrinsic clearance compared to R-MBDB HCl salt (reference compound).

Compounds (2S)-1-(2,2-Dideuterio-1,3-benzodioxol-5-yl)-N-methyl-butan-2-amine hydrochloride [S-MBDB-D2 HCl] (compound 9) and (2S)-1-(2,2-Dideuterio-1,3-benzodioxol-5-yl)-N-(trideuteriomethyl)butan-2-amine hydrochloride [S-MBDB-D5 HCl] (compound 6) exhibit significant differences in half-life and intrinsic clearance compared to S-MBDB HCl salt (reference compound). Compound (2S)-1-(1,3-Benzodioxol-5-yl)-N-(trideuteriomethyl)butan-2-amine hydrochloride [S-MBDB-ND3 HCl] (compound 3) also demonstrates differences in half-life and intrinsic clearance compared to S-MBDB HCl (reference compound).

### Example 8: Oral Bioavailability in Rats

Pharmacokinetics of test articles following a single intravenous or oral administration in rats: A pharmacokinetic (PK) study is performed in three male Sprague-Dawley (SD) rats following intravenous (IV) and oral (PO) administration of MBDB, or test deuterated MBDB, at 1 mg/kg (IV) and 10 (PO) mg/kg. Test compounds, or MBDB, are measured in plasma.

A detailed description of the in vivo methods:

### Rat Strain

Rats used in these studies are supplied by Charles River (Margate UK) and are specific pathogen free. The strain of rats is Sprague Dawley. Male rats are 175-225 g on receipt and are allowed to acclimatise for 5-7 days.

### Animal Housing

Rats are group housed in sterilised individual ventilated cages that expose the animals at all times to HEPA filtered sterile air. Animals will have free access to food and water (sterile) and will have sterile aspen chip bedding (at least once weekly). The room temperature is 22° C.+/−1° C., with a relative humidity of 60% and maximum background noise of 56 dB. Rats are exposed to 12 hour light/dark cycles.

### Treatment

Test article is diluted 10% v/v DMSO, 40% v/v PEG-400, 50% v/v Water. The test articles are administered in a dose volume of 2 mL/kg for intravenous (IV) and 5 mL/kg (PO) for oral routes of administration.

### Single IV/PO Dose Pharmacokinetics Study in Rats

Each test article is administered as a single IV bolus (via a lateral tail-vein) or a single oral gavage in cohorts of 3 rats per route. Following dose administrations, a 100 μL whole blood sample (EDTA) is collected via the tail-vein at time-points described in the table. The blood is centrifuged to separate plasma. Approximately 40 μL of plasma is dispensed per time-point, per rat, in a 96 well plate and frozen until analysis. Bioanalysis is carried out on plasma samples.

### Single IV and Oral Dose Pharmacokinetics Profile of Test Articles in Rat Plasma

| | Test | | Dose | Blood sample | No. of |
|---|---|---|---|---|---|
| Group | article | Route | (mg/kg) | collection (post dose) | rats |
| 1 | MBDB | IV | 1 | 5 min, 15 min, 30 min, | 3 |
| | | | | 1 h, 2 h, 4 h, 7 h, 24 h | |
| 2 | MBDB | PO | 10 | 15 min, 30 min, 45 min, | 3 |
| | | | | 1 h, 2 h, 4 h, 7 h, 24 h | |
| 3 | Test | IV | 1 | 5 min, 15 min, 30 min, | 3 |
| | Article | | | 1 h, 2 h, 4 h, 7 h, 24 h | |
| 4 | Test | PO | 10 | 15 min, 30 min, 45 min, | 3 |
| | Article | | | 1 h, 2 h, 4 h, 7 h, 24 h | |

### Dose Formulation Samples

Dose formulation samples are diluted in two steps with 50:50 (v/v) methanol/water to an appropriate concentration, then diluted 10:90 (v/v) with control matrix to match to the calibration standard in plasma.

### Sample Extraction Procedure

Calibration and QC standards, incurred samples, blank matrix and dose formulation samples are extracted by protein precipitation, via the addition of a bespoke acetonitrile (ACN)-based Internal Standard (IS) solution, containing several compounds and including Metoprolol and Rosuvastatin, both of which are monitored for during analysis. Following centrifugation, a 40 μL aliquot of supernatant is diluted by the addition of 80 μL water. The prepared sample extracts are analysed by LC-MS/MS.

### Example of Bioanalytical Method and Assay Information Document:

1 According to the plate layout, aliquot to wells in 0.8 mL 96-well plate (Abgene). 30 μL for Calibration, QC standards, blanks and dose formulation check.
2 Prepare Calibration and QC standards according to the assay information. Dilute dose formulation according to the assay information. Aliquot incurred samples according to the plate layout & assay information.
3 Add 90 μL of ACN internal standard and vortex mix for 5 minutes at 850 rpm
4 Centrifuge at nominally 4000 rpm for 10 minutes
6 Transfer 40 μL of supernatant into a new 0.8 mL Abgene plate.
6 Add 80 μL of water to all transferred supernatant.
7 Vortex mix for 30 seconds at 1400 rpm
8 Analyse immediately by LC-MS/MS or store at +4° C. until analysis.

### Example 9: Biological Assays and Methods

Head-Twitch Response (HTR). The head-twitch response assay is performed as is known to those of skill in the art using both male and female C57BL/6J mice (2 per treatment). The mice are obtained and were approximately 8 weeks old at the time of the experiments. Compounds were administered via intraperitoneal injection (5 mL/kg) using 0.9% saline as the vehicle. As a positive control, 5-MeO-DMT fumarate (2:1 amine/acid) was utilized. Behavior was videotaped, later scored by two blinded observers, and the results were averaged (Pearson correlation coefficient=0.93).

Serotonin and Opioid Receptor Functional Assays. Functional assay screens at 5-HT and opioid receptors are performed in parallel using the same compound dilutions and 384-well format high-throughput assay platforms. Assays assess activity at all human isoforms of the receptors, except where noted for the mouse 5-HT2A receptor. Receptor constructs in pcDNA vectors were generated from the Presto-Tango GPCR library with minor modifications. All compounds were serially diluted in drug buffer (HBSS, 20 mM HEPES, pH 7.4 supplemented with 0.1% bovine serum albumin and 0.01% ascorbic acid) and dispensed into 384-well assay plates using a FLIPR^{TETRA} (Molecular Devices). Every plate included a positive control such as 5-HT (for all 5-HT receptors), DADLE (DOR), salvinorin A (KOR), and DAMGO (MOR). For measurements of 5-HT2A, 5-HT2B, and 5-HT2C Gq-mediated calcium flux function, HEK Flp-In 293 T-Rex stable cell lines (Invitrogen) were loaded with Fluo-4 dye for one hour, stimulated with compounds and read for baseline (0-10 seconds) and peak fold-over-basal fluorescence (5 minutes) at 25° C. on the FLIPR^{TETRA}. For measurement of 5-HT6 and 5-HT7a functional assays, Gs-mediated cAMP accumulation was detected using the split-luciferase GloSensor assay in HEKT cells measuring luminescence on a Microbeta Trilux (Perkin Elmer) with a 15 min drug incubation at 25° C. For 5-HT1A, 5-HT1B, 5-HT1F, MOR, KOR, and DOR functional assays, Gi/o-mediated cAMP inhibition was measured using the split-luciferase GloSensor assay in HEKT cells, conducted similarly as above, but in combination with either 0.3 μM isoproterenol (5-HT1A, 5-HT1B, 5-HT1F) or 1 μM forskolin (MOR, KOR, and DOR) to stimulate endogenous cAMP accumulation. For measurement of 5-HT1D, 5-HT1E, 5-HT4, and 5-HT5A functional assays, P-arrestin2 recruitment was measured by the Tango assay utilizing HTLA cells expressing TEV fused-P-arrestin2, as described previously with minor modifications. Data for all assays were plotted and non-linear regression was performed using “log(agonist) vs. response” in Graphpad Prism to yield Emax and EC₅₀ parameter estimates.

5HT_{2A} Sensor Assays. HEK293T (ATCC) 5HT2A sensor stable line (sLight1.3s) is generated via lentiviral transduction of HIV-EF1α-sLight1.3 and propagated from a single colony. Lentivirus is produced using 2^{nd} generation lentiviral plasmids pHIV-EF1α-sLight1.3, pHCMV-G, and pCMV-deltaR8.2.

For the screening of the compounds, sLight1.3s cells are plated in 96-well plates at a density of 40000 24-hours prior to imaging. On the day of imaging, compounds solubilized in DMSO are diluted from the 100 mM stock solution to working concentrations of 1 mM, 100 mM and 1 μM with a DMSO concentration of 1%. Immediately prior to imaging, cells growing in DMEM (Gibco) are washed 2× with HBSS (Gibco) and in agonist mode 180 μL of HBSS or in antagonist mode 160 μL of HBSS is added to each well after the final wash. For agonist mode, images are taken before and after the addition of the 20 μL compound working solution into the wells containing 180 μL HBSS. This produces final compound concentrations of 100 mM, 10 mM and 100 nM with a DMSO concentration of 0.1%. For antagonist mode, images are taken before and after addition of 20 μL of 900 nM 5-HT and again after 20 μL of the compound working solutions to produce final concentrations of 100 nM for 5HT and 100 mM, 10 mM and 100 nM for the compounds with a DMSO concentration of 0.1%. Each compound is tested in triplicate (3 wells) for each concentration (100 mM, 10 mM and 100 nM). Additionally, within each plate, 100 nM 5HT and 0.1% DMSO controls are also imaged.

Imaging is performed using the Leica DMi8 inverted microscope with a 40× objective using the FITC preset with an excitation of 460 nm and emission of 512-542 nm. For each well, the cellular membrane where the 5HT2A sensor is targeted is autofocused using the adaptive focus controls and 5 images from different regions within the well were taken with each image processed from a 2×2 binning.

For data processing, the membranes from each image are segmented and analyzed using a custom algorithm written in MATFAB producing a single raw fluorescence intensity value. For each well the 5 raw fluorescence intensity values generated from the 5 images are averaged and the change in fluorescence intensity (dFF) is calculated as:

dFF=(Fₛₐₜ−Fₐₚₒ)/Fₐₚₒ

For both agonist and antagonist modes, the fluorescence intensity values before compound addition in FIBSS only are used as the Fₐₚₒ values while the fluorescence intensity values after compound addition are used as the Fₛₐₜ values.

For agonist mode, data are as percent activation relative to 5HT, where 0 is the average of the DMSO wells and 100 is the average of the 100 μM 5HT wells. For antagonist mode, the inactivation score is calculated as:

Inactivation score=(dFFF(Compound+5HT)−dFF(5HT))/dFF(5HT)

Plasticity Effects: Treatment of rat embryonic cortical neurons with compounds of Formulas I and II is evaluated for increased dendritic arbor complexity at 6 days in vitro (DIV6) as measured by Sholl analysis. The effect of the present compounds on dendritic growth can be determined to be 5-HT2A-dependent, if pretreatment with ketanserin-a 5-HT2A antagonist-inhibits their effects.

In addition to promoting dendritic growth, the present compounds also are evaluated for increased dendritic spine density to a comparable extent as ibogaine in mature cortical cultures (DIV20). The effects of the compounds on cortical dendritic spine dynamics in vivo using transcranial 2-photon imaging is assessed. First, spines are imaged on specific dendritic loci defined by their relation to blood vessel and dendritic architectures. Next, the animals are systemically administered vehicle, a compound of the present invention, or the hallucinogenic 5-HT2A agonist 2,5-dimethoxy-4-iodoamphetamine (DOI). After 24 h, the same dendritic segments are re-imaged, and the number of spines gained or lost is quantified. Examples of the presently disclosed compounds increase spine formation in mouse primary sensory cortex, suggesting that the present compounds support neuronal plasticity.

As increased cortical structural plasticity in the anterior parts of the brain mediates the sustained (>24 h) antidepressant-like effects of ketamine and play a role in the therapeutic effects of 5-HT2A agonists, the impact of the present compounds on forced swim test (FST) behavior is evaluated. First, a pretest is used to induce a depressive phenotype. Compounds are administered 24 h after the pre-test, and the FST is performed 24 h and 7 d post compound administration. Effective compounds of the invention, like ketamine, significantly reduced immobility 24 h after administration.

Dendritogenesis Assays. Compounds disclosed herein are evaluated of their ability to increase dendritic arbor complexity in cultures of cortical neurons using a phenotypic assay. Following treatment, neurons are fixed and visualized using an antibody against MAP2—a cytoskeletal protein localized to the somatodendritic compartment of neurons. Sholl analysis is then performed, and the maximum number of crossings (Nₘₐₓ) was used as a quantitative metric of dendritic arbor complexity. For statistical comparisons between specific compounds, the raw Nₘₐₓ values are compared. Percent efficacies are determined by setting the Nₘₐₓ values for the vehicle (DMSO) and positive (ketamine) controls equal to 0% and 100%, respectively.

Animals. For the dendritogenesis experiments, timed pregnant Sprague Dawley rats are obtained. For the head-twitch response assay, male and female C57BL/6J mice are obtained.

Dendritogenesis—Sholl Analysis. Dendritogenesis experiments are performed following a previously published methods with slight modifications. Neurons are plated in 96-well format (200 μL of media per well) at a density of approximately 15,000 cells/well in Neurobasal (Life Technologies) containing 1% penicillin-streptomycin, 10% heat-inactivated fetal bovine serum, and 0.5 mM glutamine. After 24 h, the medium is replaced with Neurobasal containing 1× B27 supplement (Life Technologies), 1% penicillin-streptomycin, 0.5 mM glutamine, and 12.5 μM glutamate. After 3 days in vitro (DIV3), the cells are treated with compounds. All compounds tested in the dendritogenesis assays are treated at 10 μM. Stock solutions of the compounds in DMSO are first diluted 100-fold in Neurobasal before an additional 10-fold dilution into each well (total dilution=1:1000; 0.1% DMSO concentration). Treatments are randomized. After 1 h, the media is removed and replaced with new Neurobasal media containing 1× B27 supplement, 1% penicillin-streptomycin, 0.5 mM glutamine, and 12.5 mM glutamate. The cells are allowed to grow for an additional 71 h. At that time, neurons are fixed by removing 80% of the media and replacing it with a volume of 4% aqueous paraformaldehyde (Alfa Aesar) equal to 50% of the working volume of the well. Then, the cells are incubated at room temperature for 20 min before the fixative is aspirated and each well washed twice with DPBS. Cells are permeabilized using 0.2% Triton X-100 (ThermoFisher) in DPBS for 20 minutes at room temperature without shaking. Plates are blocked with antibody diluting buffer (ADB) containing 2% bovine serum albumin (BSA) in DPBS for 1 h at room temperature. Then, plates are incubated overnight at 4° C. with gentle shaking in ADB containing a chicken anti-MAP2 antibody (1:10,000; EnCor, CPCA-MAP2). The next day, plates are washed three times with DPBS and once with 2% ADB in DPBS. Plates are incubated for 1 h at room temperature in ADB containing an anti-chicken IgG secondary antibody conjugated to Alexa Fluor 488 (Life Technologies, 1:500) and washed five times with DPBS. After the final wash, 100 μL of DPBS is added per well and imaged on an ImageXpress Micro XL High-Content Screening System (Molecular Devices, Sunnyvale, Calif.) with a 20× objective. Images are analyzed using ImageJ Fiji (version 1.51 W). First, images corresponding to each treatment are sorted into individual folders that are then blinded for data analysis. Plate controls (both positive and negative) are used to ensure that the assay is working properly as well as to visually determine appropriate numerical values for brightness/contrast and thresholding to be applied universally to the remainder of the randomized images. Next, the brightness/contrast settings are applied, and approximately 1-2 individual pyramidal-like neurons per image (i.e., no bipolar neurons) are selected using the rectangular selection tool and saved as separate files. Neurons are selected that do not overlap extensively with other cells or extend far beyond the field of view.

In Vivo Spine Dynamics. Male and female Thy1-GFP-M line mice (n=5 per condition) are purchased from The Jackson Laboratory (JAX #007788) and maintained. In vivo transcranial two-photon imaging and data analysis are performed as previously described. Briefly, mice are anesthetized with an intraperitoneal (i.p.) injection of a mixture of ketamine (87 mg/kg) and xylazine (8.7 mg/kg). A small region of the exposed skull is manually thinned down to 20-30 pm for optical access. Spines on apical dendrites in mouse primary sensory cortices are imaged using a Bruker Ultima IV two-photon microscope equipped with an Olympus water-immersion objective (40×, NA=0.8) and a Ti:Sapphire laser (Spectra-Physics Mai-Tai, excitation wavelength 920 nm). Images are taken at a zoom of 4.0 (pixel size 0.143×0.143 pm) and Z-step size of 0.7 pm. The mice receive an i.p. injection (injection volume=5 mL/kg) of DOI (10 mg/kg) or TBG (50 mg/kg) immediately after they recovered from anesthesia given prior to the first imaging session. The animals are re-imaged 24 h after drug administration. Dendritic spine dynamics were analyzed using ImageJ. Spine formation and elimination were quantified as percentages of spine number on day 0.

Forced Swim Test (FST). Male C57/BL6J mice (9-10 weeks old at time of experiment) are obtained. After 1 week in the vivarium each mouse is handled for approximately 1 minute by the experimenter for 3 consecutive days leading up to the first FST. All experiments are carried out by the same experimenter who performs handling. During the FST, mice undergo a 6 min swim session in a clear Plexiglas cylinder 40 cm tall, 20 cm in diameter, and filled with 30 cm of 24±1° C. water. Fresh water is used for every mouse. After handling and habituation to the experimenter, drug-naive mice first undergo a pretest swim to more reliably induce a depressive phenotype in the subsequent FST sessions. Immobility scores for all mice are determined after the pre-test and mice are randomly assigned to treatment groups to generate groups with similar average immobility scores to be used for the following two FST sessions. The next day, the animals receive intraperitoneal injections of experimental compounds (20 mg/kg), a positive control (ketamine, 3 mg/kg), or vehicle (saline). The animals were subjected to the FST 30 mins after injection and then returned to their home cages. All FSTs are performed between the hours of 8 am and 1 pm. Experiments are video-recorded and manually scored offline. Immobility time defined as passive floating or remaining motionless with no activity other than that needed to keep the mouse's head above water is scored for the last 4 min of the 6 min trial.

Statistical analysis. Treatments are randomized, and data are analyzed by experimenters blinded to treatment conditions. Statistical analyses are performed using GraphPad Prism (version 8.1.2). The specific tests are, F-statistics and degrees of freedom. All comparisons are planned prior to performing each experiment. For dendritogenesis experiments a one way ANOVA with Dunnett's post hoc test is deemed most appropriate. Ketamine was included as a positive control to ensure that the assay is working properly.

Alcohol Use Disorder Model: To assess the anti-addictive potential of the present compounds, an alcohol drinking paradigm that models heavy alcohol use and binge drinking behavior in humans is employed. Using a 2-bottle choice setup (20% ethanol (v/v), EtOH vs. water, H₂O), mice are subjected to repeated cycles of binge drinking and withdrawal over the course of 7 weeks.

This schedule results in heavy EtOH consumption, binge drinking-like behavior, and generates blood alcohol content equivalent to that of human subjects suffering from alcohol use disorder (AUD). Next, compounds of the invention are administered via intraperitoneal injection 3 h prior to a drinking session, and EtOH and H₂O consumption is monitored. Effective compounds of the invention robustly reduce binge drinking during the first 4 h, decreasing EtOH consumption. With exemplary compounds, consumption of ethanol is lower for at least two days following administration with no effect on water intake. Efficacy in this assay suggests the present compounds are useful for the treatment of AUD.

### Example 10: Zeromaze Study

### Background

The rat zero-maze model is a refined alternative to the plus-maze, the most widely used animal model of anxiety, and consists of an elevated annular platform, divided equally into four quadrants. Two opposite quadrants are enclosed by Perspex walls on both the inner and the outer edges of the platform, while the remaining two opposite quadrants are open being enclosed only by a Perspex “lip”. Animals will show a preference for the closed areas, and avoidance of the open sections is assumed to stem from a rodent's natural aversion to open, exposed spaces. A reduction in the amount of activity on the open areas is considered to reflect an increase in anxiety. The ethologically-based behavior stretched attend postures (SAP) from closed to open quadrants is assessed as an index of anxiety. Increase in SAPs is indicative of an anxiogenic effect and decreases in SAPs is indicative of an anxiolytic effect.

Shepherd, J K; Grewel, S S; Fletcher, A; Bill, D J; Dourish, C T (1994) Behavioural and pharmacological validation of the elevated “zero-maze” as an animal model of anxiety. Psychopharmacol., 116:56-64.

### Animals

Male Sprague-Dawley 200-250 g (Envigo UK) rats were used. Animals were group-housed (5 per cage; cage size: 40×40×20 cm) in a temperature-controlled environment (22±2° C.), under a 12 h light-dark cycle (lights on: 08:00 hours) for one week prior to testing. Food and water were freely available. Number of animals per group=5. Animals were moved into the experimental room 16-24 hours before testing.

### Apparatus

The elevated 0-maze comprises a black Perspex annular platform (105 cm diameter, 10 cm width) elevated to 65 cm above ground level, divided equally into four quadrants. Two opposite quadrants are enclosed by clear red Perspex walls (27 cm high) on both the inner and outer edges of the platform, while the remaining two opposite quadrants are surrounded only by a Perspex “lip” (1 cm high) which serves as a tactile guide to animals on these open areas.

### Procedure

Subjects were weighed and tail marked before being injected. After a specified pretreatment time, subjects were placed in a closed quadrant and a 5-min test period were recorded on videotape for subsequent analysis. The maze was cleaned with 5% methanol/water solution and dried thoroughly between test sessions. Behavioural measures comprise percentage time spent on the open areas (% TO) and frequency of stretched attend postures (SAP) from closed to open quadrants. Since the control groups were all treated identically with the same vehicle these were combined to increase power. The Chlordiazepoxide groups were also treated identically with the same dose so these were also combined to increase power. Animals are scored as being in the open area when all four paws were in an open quadrant and in the closed area only when all four paws have passed over the open-closed divide. All testing were carried out between 9.00 and 16.00 hours.

### Formulation:

IP: Rac-MBDB (tosylate salt with 54.6% free base content) was formulated in Vehicle 1 (Saline) for injection to concentrations of 0.5, 1, 2, 3 and 6 mg/mL to provide doses of 2.5, 5, 10, 15 and 30 mg/kg when administered ip in 5 mL/kg dosing volumes.

IP: R-MBDB (tosylate salt with 54.6% free base content) was formulated in Vehicle 1 (Saline) for injection to concentrations of 0.5, 1, 2, 3 and 6 mg/mL to provide doses of 2.5, 5, 10, 15 and 30 mg/kg when administered ip in 5 mL/kg dosing volumes IP: S-MBDB (tosylate salt with 54.6% free base content) was formulated in Vehicle 1 (Saline) for injection to concentrations of 0.5, 1, 3 and 6 mg/mL to provide doses of 2.5, 5, 15 and 30 mg/kg when administered ip in 5 mL/kg dosing volumes.

Chlordiazepoxide was formulated in Vehicle 1 (saline) to a concentration of 1.2 mg/mL to provide a dose of 6 mg/kg when administered ip in 5 mL/kg dosing volumes.

### Effect of Administration of Rac-MBDB and Chlordiazepoxide on Behavior in a Rat 0-Maze Study

35 male Sprague-Dawley rats in treatment groups of 5, were intraperitoneally dosed with either Vehicle 1 (saline) or Rac-MBDB at 1 of 5 dose levels (2.5, 5, 10, 15 & 30 mg/kg) or chlordiazepoxide (6 mg/kg) in 5 mL/kg injection volumes. Thirty min later at T=0, rats were individually placed in a closed arm of the zero-maze and behavior assessed by a “blind” observer using remote video monitoring over the subsequent 5 min. The animals were then removed and the maze carefully wiped with 5% methanol/water solution before the next test was begun.

### Synopsis of Testing Schedule Rac-MBDB and Chlordiazepoxide in the Rat Elevated Zero Maze Model of Anxiety.

| | Rat Strain | 0.5 Pretest in Veh 1 | T = 0 |
|---|---|---|---|
| n | & sex | 5 mL/kg ip | Zero-maze |
| 5 | Male SD | Vehicle 1 | Test |
| 5 | Male SD | Rac-MBDB 2.5 mg/kg | Test |
| 5 | Male SD | Rac-MBDB 5 mg/kg | Test |
| 5 | Male SD | Rac-MBDB 10 mg/kg | Test |
| 5 | Male SD | Rac-MBDB 15 mg/kg | Test |
| 5 | Male SD | Rac-MBDB 30 mg/kg | Test |
| 5 | Male SD | CDP 6 mg/kg | Test |

### Effect of Administration of R-MBDB and Chlordiazepoxide on Behavior in a Rat 0-Maze Study

35 male Sprague-Dawley rats in treatment groups of 5, were intraperitoneally dosed with either Vehicle 1 (saline) or R-MBDB at 1 of 5 dose levels (2.5, 5, 10, 15 & 30 mg/kg) or chlordiazepoxide (6 mg/kg) in 5 mL/kg injection volumes. Thirty min later at T=0, rats were individually placed in a closed arm of the zero-maze and behavior was assessed by a “blind” observer using remote video monitoring over the subsequent 5 min. The animal were then removed and the maze carefully wiped with 5% methanol/water solution before the next test was begun.

### Synopsis of Testing Schedule R-MBDB and Chlordiazepoxide in the Rat Elevated Zero Maze Model of Anxiety.

| | Rat Strain | 0.5 Pretest in Veh 1 | T = 0 |
|---|---|---|---|
| n | & sex | 5 mL/kg ip | Zero-maze |
| 5 | Male SD | Vehicle 1 | Test |
| 5 | Male SD | R-MBDB 2.5 mg/kg | Test |
| 5 | Male SD | R-MBDB 5 mg/kg | Test |
| 5 | Male SD | R-MBDB 10 mg/kg | Test |
| 5 | Male SD | R-MBDB 15 mg/kg | Test |
| 5 | Male SD | R-MBDB 30 mg/kg | Test |
| 5 | Male SD | CDP 6 mg/kg | Test |

### Effect of Administration of S-MBDB and Chlordiazepoxide on Behavior in a Rat 0-Maze Study

35 male Sprague-Dawley rats in treatment groups of 5, were intraperitoneally dosed with either Vehicle 1 (saline) or S-MBDB at 1 of 5 dose levels (2.5, 5, 10, 15 & 30 mg/kg) or chlordiazepoxide (6 mg/kg) in 5 mL/kg injection volumes. Thirty min later at T=0, rats were individually placed in a closed arm of the zero-maze and behavior was assessed by a “blind” observer using remote video monitoring over the subsequent 5 min. The animals were then removed and the maze carefully wiped with 5% methanol/water solution before the next test was begun.

### Synopsis of Testing Schedule S-MBDB and Chlordiazepoxide in the Rat Elevated Zero Maze Model of Anxiety.

| | Rat Strain | 0.5 Pretest in Veh 1 | T = 0 |
|---|---|---|---|
| n | & sex | 5 mL/kg ip | Zero-maze |
| 5 | Male SD | Vehicle 1 | Test |
| 5 | Male SD | S-MBDB 2.5 mg/kg | Test |
| 5 | Male SD | S-MBDB 5 mg/kg | Test |
| 5 | Male SD | S-MBDB 10 mg/kg | Test |
| 5 | Male SD | S-MBDB 15 mg/kg | Test |
| 5 | Male SD | S-MBDB 30 mg/kg | Test |
| 5 | Male SD | CDP 6 mg/kg | Test |

### Statistical Analysis

### Drug Versus Vehicle Treatments

For each study, a 1-way ANOVA was conducted across vehicle, CDP, and drug treatment groups. Each group was compared to the vehicle group and a p-value for treatment determined by Fishers Least Significant Difference (LSD) test. This analysis was performed in GraphPad Prism (Version 9).

### Results

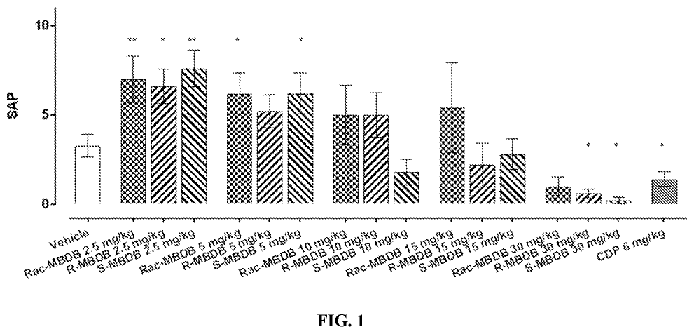

The positive control CDP did not show significance over vehicle on the percentage of time in the open arms (% TO) measure so we could not use % TO in this experiment as a measure to examine the effects of MBDB. We then evaluated SAPs as the primary measure (Shepherd 1994). Shepherd describes using SAPs in cases where % TO does not show significance. In this analysis the positive control CDP did show a significant reduction in SAPs show in FIG. 1 so this measure was expanded to the MBDB groups.

### Discussion

The results show that the 30 mg/kg dose S-MBDB, and R-MBDB both decreased the frequency of SAPs as effectively as the benzodiazepine chlordiazepoxide and racemic MBDB showed a trend toward significance (FIG. 1). This shows that at a sufficient dose, racemic MBDB, S-MBDB, and R-MBDB are each effective anxiolytics and supports their development in these indications. This is the first in vivo data showing MBDB is effective in these indications. However, there were some unexpected findings that show S-MBDB and R-MBDB are surprisingly not equivalent in regard to side effects that further inform dose selection for their therapeutic use in humans.

First, for all forms of MBDB, the lowest dose tested (2.5 mg/kg) showed a decrease SAPs vs placebo (FIG. 1). This indicates that this low dose of MBDB had an anxiogenic effect. This effect continued at the next dose level 5 mg/kg, for all three forms of MBDB and reached significance for both racemic MBDB and S-MBDB. This trend toward increased anxiety continued at 10 mg/kg for racemic MBDB and R-MBDB which produced a consistent level of SAPs similar to the 5 mg/kg, however, in contrast to racemic and R-MBDB, S-MBDB showed a rapid reduction in total SAPs at this dose level. This suggests that S-MBDB may have advantages over racemic MBDB and R MBDB. This effect continued at 15 mg/kg where both R MBDB and S MBDB showed a reduction in the number of SAPs compared to lower doses while racemic MBDB did not. However, the magnitude of the decrease in SAPs was not as great as chlordiazepoxide at this dose. Finally, at 30 mg/kg R MBDB and S MBDB both significantly reduced the number of SAPs, however the magnitude of the reduction was greatest with S MBDB, again providing support that S MBDB may me more effective at reducing anxiety at high doses and less likely to be anxiogenic at low doses than racemic MBDB and R MBDB. This data indicates that for MBDB, low doses can paradoxically increase anxiety and that this anxiogenic effect shifts to an anxiolytic effect at doses that are sufficiently high to induce a therapeutic effect.

There was an even stronger dose dependent anxiogenic effect observed with racemic-MBDB and R MBDB than with S MBDB. The data show that the lowest doses of MBDB induce an anxiogenic effect with this effect switching to an anxiolytic effect as the dose is increased. However, when considering the magnitude of the reduction in SAPs S-MBDB showed a switch to an anxiolytic effect at the 10 mg/kg range while R-MBDB showed this at 15 mg/kg and racemic MBDB only at 30 mg/kg. This suggests that S MBDB has a greater therapeutic index than racemic MBDB and R-MBDB and has a more reduced range of doses that could increase anxiety when compared to racemic MBDB and R-MBDB. Additionally, since the total reduction in SAPs was lowest with S-MBDB, it may have a greater overall therapeutic effect than racemic MBDB and R-MBDB. This indicates that the anxiogenic side effects seen with lower doses of racemic MBDB may be due to the increased anxiogenic effects of R-MBDB.

There are several critical implications of this finding. The first is that patients treated with any form of MBDB must receive a dose high enough to reach the anxiolytic threshold since lower doses may cause anxiety as a side effect and result in worsening of the disorder being treated and that this effect may be particularly severe with racemic MBDB and R-MBDB. This could have especially severe implications for anxiety disorders or depressive disorders including post-traumatic stress disorder, generalized anxiety disorder, panic disorder, major depressive disorder, or treatment resistant depression. All of these indications are associated with an increased level of anxiety as a major symptom. In these cases, a drug-induced increase in anxiety due to improper dosing of MBDB could have severe side effects on patients and worsen their underlying disorder. The data presented herein show that patients treated with a racemic MBDB or S-MBDB must be especially careful to titrate up to the therapeutic dose to avoid the anxiogenic effects and to reach the anxiolytic effect level. The data show that in some embodiments a Risk Evaluation and Mitigation Strategy (REMS) program should be utilized so that patients treated with MBDB should undergo an initial dose titration to determine the effective range specific to that patient. This dose titrating protocol would decrease the side effects related to underdosing MBDB.

The data also inform Phase 2 and Phase 3 clinical trial design. Clinical trials for neurological and psychiatric disorders often include one or more low dose arms to show a dose dependent effect of the full dose on the disease of interest. However, this data shows that MBDB should only be dosed at the full effective dose and a low dose arm should not be included as a comparator as this may lead to harmful side effects on the patients. This data shows that studies of MBDB should only use inactive matched placebo or a different standard of care therapeutic as a control. In clinical trials MBDB should only be dosed at its effective dose range to avoid harmful side effects to the patients. This would be especially critical in clinical studies of anxiety disorders or depression including post-traumatic stress disorder, generalized anxiety disorder, panic disorder, major depressive disorder, or treatment resistant depression where increased anxiety could worsen the underlying disorder and lead to potentially devastating effects on the patients.

The data show that there is an advantage of S-MBDB which has a greater therapeutic index and a lower range of doses that would produce anxiogenic side effects. In some embodiments, a clinician treating a patient with S-MBDB does not need to utilize a specific dose titration protocol to reduce anxiogenic effects. In some embodiments clinical studies of S-MBDB have a greater safety margin and are able to use lower doses in different arms of the study to demonstrate a dose dependent effect on the disease of interest. In some embodiments, S-MBDB allows greater flexibility in clinical trial design including the safe use of a low dose active comparator to reduce expectancy bias. In some embodiments, S-MBDB would be preferred to racemic MBDB or R-MBDB to treat patients with anxiety or depressive disorders including post-traumatic stress disorder, generalized anxiety disorder, panic disorder, major depressive disorder, or treatment resistant depression. In some embodiments S-MBDB is a safer alternative to racemic MBDB or R-MBDB for the treatment of neurological and psychiatric disorders.

### Example 11: MBDB Dose Titration Risk Evaluation and Mitigation Strategy (REMS) Protocol

### General Information on MBDB Treatment Session

Initial MBDB dosing and subsequent dosing adjustments must be done under the supervision of a qualified healthcare professional in a clinic or inpatient setting. The patient must remain under supervision of the healthcare professional for at least 6 hours and up to approximately 24 hours after the final MBDB dose adjustment. The patient will be assessed periodically during the session for anxiety and other effects of MBDB. Dose adjustments within a MBDB treatment session will be based on changes from baseline levels of anxiety. Postdose anxiety measurement timing and duration of observation after dosing are shown here as adapted from PiHKAL 1991:

### Duration of Effects of MBDB

| Effects | Time After Dose |
|---|---|
| Onset | 20 min-30 min |
| Plateau | 60 min-90 min |
| Coming Down | ~90 min |
| Duration | 4 hours-6 hours |

MBDB dosing is shown here:

### MBDB Dosages

| Drug Activity Level | Oral MBDB Dose |
|---|---|
| Threshold | 60 mg-120 mg |
| Light | 120 mg-150 mg |
| Common | 150 mg-250 mg |
| Strong | 250 mg-350 mg |
| Very Strong | ≥350 mg |

### Predose Assessment

The patient's baseline level of anxiety will be measured and recorded.

### Initial MBDB Dosing

The patient will receive an initial single oral dose of MBDB in the range of approximately 180 mg-210 mg based on oral doses reported as producing moderate effects (PiHKAL 1991).

### Postdose Assessment

Change from baseline anxiety level will be measured at approximately 0.75 to 1 hours after dosing based on reported time to achieve peak effects (PiHKAL 1991).

### MBDB Dose Adjustment

MBDB effects have been maintained by taking a larger initial dose followed by smaller doses (30 mg to 100 mg p.o.). Re-dose of one-third to one-half the initial dose usually prolongs duration (PiHKAL 1991). Accordingly, the dose of MBDB will be adjusted based on change from baseline in anxiety as follows:

### MBDB Dose Adjustment

| Change from | |
|---|---|
| Baseline Anxiety | MBDB Dose Adjustment |
| Increased | Increase dose 30%-50% and reassess anxiety in |
| | approximately 1 hour |
| No Change | Increase dose 30%-50% and reassess anxiety in |
| | approximately 1 hour |
| Decreased | Maintain dose if therapeutic effect achieved |
| | or |
| | Increase to a maximum of 350 mg total dose to |
| | optimize therapeutic effect |

### MBDB Discontinuation

The patient will be observed for at least 6 hours after final MBDB dose is administered.

The patient may be confined to the inpatient unit for prolonged observation up to approximately 24 hours after last MBDB dose if indicated based on persistent effects.

Anxiety that appears after the final MBDB titration dose is administered can be managed with an appropriate anxiolytic agent. If this is necessary, the patient must remain under observation and undergo periodic reassessment until the supervising healthcare professional determines the patient can be discharged from care.

### Example 7: A Double-Blind, Randomized, Placebo-Controlled Clinical Trial of MBDB-Assisted Psychotherapy in PTSD

A multicenter, randomized, double-blind, placebo-controlled trial is conducted to assess the efficacy and safety of MBDB-assisted psychotherapy versus psychotherapy with placebo control in participants diagnosed with at least moderate post-traumatic stress disorder (PTSD).

### Rationale

PTSD is a debilitating and often times chronic disorder associated with profound mental, physical, occupational, and functional impairment. PTSD can develop due to exposure to a traumatic event or persistent or recurring threats to an individual. Studies indicate that approximately 10% of individuals exposed to a traumatic event eventually go on to be diagnosed with PTSD (American Psychiatric Association. Diagnostic and statistical manual of mental disorders, 5^{th} edition, 2013). PTSD is a complex psychiatric disorder characterized by symptom heterogeneity including avoidance of trauma-related material, emotional blunting and distancing, hyper-vigilance, hyper-arousal, persistent negative alterations in mood, persistent alterations in cognition, disturbing thoughts, disruptions in sleep and/or dreams, and physical or mental distress. Symptoms can be severe and long lasting. Although this symptom heterogeneity may suggest a wide spectrum of separate disturbances, emotional dysregulation is considered to be a core component of this disorder. Particularly germane to the pathogenesis and progression of PTSD, emotional dysregulation in affected individuals is believed to give rise to observable and measurable features such as presence of hypervigilance and attentional biases, enhanced startle response, hyper-arousal, apathetic feeling or emotional numbness, irritability, enhanced memories associated with traumatic events, difficulty in discerning danger versus safety, a generalization of fear, and avoidance of reminders of trauma. Emotional dysregulation may be defined and also measured by elevated emotional reactivity based on abnormal detection or appraisal of emotional triggers involving bottom-up sensory detection and neuronal processing. Biochemical alterations found in individuals diagnosed with PTSD suggest abnormalities in the hypothalamic-pituitary-adrenal (HPA) axis. The HPA axis is known to regulate reactions to stress and controls significant aspects of the neuroendocrine system impacting many homeostatic systems in the body. In a typical flight-or-flight response in a healthy individual, catecholamine and cortisol levels detected in urine rise after exposure to a stressor. In PTSD, many individuals show a low secretion of cortisol and high secretion of catecholamine in response to a stressor indicating a change in catecholamine to cortisol ratio in the urine. More evidence that the HPA axis is impacted in PTSD is found in elevated levels of catecholamines and corticotropin-releasing factor in the brain of many affected individuals.

The initiation and/or maintenance of emotional dysregulation in PTSD may be due to abnormalities in top-down control of emotional responses indicating that cognitive influences and higher order representations may impinge on information and emotional processing. Certainly, some aspects of abnormalities in neuronal processing in PTSD occur either implicitly (e.g., unconsciously) or explicitly (e.g., consciously) indicating involvement of distinct cognitive processes. Exaggerated responses in the amygdala and insular cortex have been demonstrated in meta-analyses in PTSD pathology, as have decreases in activity in other brain regions including the anterior cingulate cortex and aspects the prefrontal cortex including the ventromedial prefrontal cortex. In addition to changes in patterns of neuronal activity in individuals with PTSD, several neuroanatomical changes in PTSD have also been demonstrated. A reduction of total brain volume, intracranial volume, and the volumes in regions such as the hippocampus (particularly localized to the CA3 and dentate gyrus regions), insular cortex, and anterior cingulate cortex have been indicated in occurring in some individuals with PTSD through meta-analyses of structural MRI studies. Animal studies have shown that severe chronic stress leads to atrophy of apical dendrites in the CA3 region of the hippocampus, reduced hippocampus neurogenesis, and elevated granule cell death in the dentate gyrus due to elevated levels of glucocorticoids (Gould E. and Tanapat. (1999). Stress and hippocampal neurogenesis. Biol. Psychiatry 46, 1472-1479.) Connections between brain areas such as the amygdala, hippocampus, prefrontal cortex, and hypothalamus can facilitate activation of the HPA axis to illustrate interactions between brain regions with structural changes and affected biochemical regulatory systems in PTSD.

MBDB is a synthetic analog of the psychedelic phenethylamine class of compounds known to act as a mixed reuptake inhibitor/releasing agent of serotonin, norepinephrine, and dopamine and administration of MBDB can produce acute modulations of neurotransmission.

MBDB administration also has indirect effects on neurohormone release. MBDB can function as a psychoplastogen promoting neuronal growth, modulating neuronal connectivity, and regulating neuronal plasticity through longer term neuronal changes. The combined neurobiological effects of MBDB administration on individuals reduce fear of emotional injury or distress, enhance introspection and communication, and increase empathetic feelings and compassion. Additionally, MBDB may serve to enhance fear extinction. These combined effects may yield acute and longer-term productive psychological states to enhance behavioral or cognitive-behavioral therapies. MBDB administration may enhance neuronal function at the biochemical and cellular levels to generate or restore favorable neural network pathways and connectivity to increase behavioral or cognitive-behavioral therapy productiveness.

### Study Design

This multicenter, randomized, double-blind, placebo-controlled trial is conducted at various sites in the United States with IRB approval from each study site. A flexible dose of MBDB hydrochloride salt or placebo, followed by a supplemental half-dose unless contraindicated by patient's previous response or medical history, is also administered during the Treatment Period with psychotherapy in at least 3 blinded monthly Experimental Sessions. The Supplemental Dose extends the duration of drug effects on the participants during an Experimental Session. MBDB test groups are further subdivided into specific groups receiving only racemic MBDB hydrochloride salt, S-MBDB hydrochloride salt, or R-MBDB hydrochloride salt. An optional Risk Evaluation and Mitigation Strategy (REMS) Protocol may be implemented for the racemic MBDB, S-MBDB, R-MBDB, and placebo-groups. The Treatment Period lasts for approximately 12 weeks. During the Treatment Period, each Experimental Session is followed by three Intervening Sessions of non-drug psychotherapy. Each Experimental Session involves an overnight stay. The Primary Outcome measure, the change in Clinician Administered PTSD Scale for DSM-5 (CAPS-5), is determined by a blinded Independent Rater (IR) pool multiple times throughout the study. The study consists of separate periods for each participant. Initially, prospective participants undergo a Screening Period involving an initial eligibility assessment, a medical history intake, informed consent, and enrollment of eligible participants. Next, a Preparation Period is undertaken for enrolled participants involving medication tapering and clinical baseline assessments to confirm each participant meets enrollment criteria. As part of the Preparation Period, a detailed assessment of co-morbidities to PTSD is recorded. Participants may remain on prescribed courses of selective serotonin reuptake inhibitor (SSRI) or serotonin and norepinephrine reuptake inhibitor (SNRI) treatment. Dosages and/or frequency of administration of a prescribed SSRI or SNRI may be adjusted to fit within study parameters. Participants may be required to taper a prescribed course of medication in order to maintain eligibility within the study. The Treatment Period consists of three monthly Experimental Sessions and associated Intervening Sessions of integrative behavioral psychotherapy. The Treatment Period lasts approximately 12 weeks. Following the Treatment Period is a Follow-up Period and Study Conclusion. During the Follow-up Period and Study Conclusion, participants complete 4 weeks with no study visits, followed by a Study Conclusion visit.

Screening Period - from initial consent to beginning of enrollment (approx. 4 weeks)

| Study Visit | | Visit Timing | Description |
|---|---|---|---|
| Screening | Screening | Several visits | Informed consent obtained and assessment |
| | | taking place | measures of pre-study medications, complete |
| | | 5-30 days | personal and family medical history and all |
| | | after initial | assessed screening measures undertaken. These |
| | | phone call | measures may include any of: PTSD checklist for |
| | | screen | DSM-5 (PCL-5), Columbia-Suicide Severity |
| | | | Rating Scale (C-SSRS), Montgomery-Asberg |
| | | | Depression Rating Scale (MADRS), Hamilton |
| | | | Depression Rating Scale (HAM-D), Hamilton |
| | | | Anxiety Rating Scale (HAM-A), General Anxiety |
| | | | Disorder-7 (GAD-7), Beck Anxiety Inventory |
| | | | (BAI), Impact of Events Scale (IES), State-Trait |
| | | | Anxiety Inventory (STAI), Edinburgh Postnatal |
| | | | Depression Scale (EPDS), Clinical Global |
| | | | Impressions Scale (CGI-I), Epworth Sleepiness |
| | | | Scale (ESS), and Pittsburgh Sleep Quality Scale. |
| | | | Medical providers are contacted and medical |
| | | | records and laboratory results are obtained. All |
| | | | results and records are reviewed along with |
| | | | interview notes. If eligible, results of Life Events |
| | | | Checklist for DSM-5 (LEC-5) and Structured |
| | | | Clinical Interview for DSM-5 Personality |
| | | | Questionnaire (SCID-5-SPQ) are forwarded to IR. |
| | IR | 2-10 days | Initial eligibility after PCL-5 and initial eligibility |
| | Screening | after initial | are reviewed. Next, IR conducts a since last visit |
| | | eligibility | C-SSRS, SCID-5-PD, Dissociative Disorders |
| | | determined | Interview Schedule (DDIS), and/or International |
| | | during | Neuropsychiatric Interview (MINI). Results of IR |
| | | Screening | assessment confirmed over Preparatory Period. |
| | | Period | |
| Enrollment | Enrollment | 1-14 days | Prior to enrollment, all screening measures are |
| | | after IR | reviewed and any clarification needed with |
| | | Screening | participant is completed by telephone interview. If |
| | | | enrolled, and if it has been determined to taper an |
| | | | ongoing medication, begin a tapering treatment |
| | | | plan of at least 5 half-lives plus at least 5 days for |
| | | | stabilization. Begin collection of Adverse Events |
| | | | (AE). |

Preparation Period (between 1-12 weeks)

| Study Visit | | Visit Timing | Description |
|---|---|---|---|
| Preparatory | Preparatory | Undertaken 0- | Schedule visit timing according to medication |
| Period | Session 1 | 14 days | tapering needs. Schedule calls in between |
| | | post- | visits for safety concerns, tapering questions, |
| | | enrollment | or other issues related to medical history. |
| | | | Confirm enrollment. |
| | Preparatory | Undertaken 2- | Schedule upcoming visits if medication |
| | Session 2 | 21 days | tapering is not needed or is already completed. |
| | | following | If still tapering, schedule additional telephone |
| | | Preparation | call for continuing assessment of readiness to |
| | | Session 1 | enter study. |
| | Taper | 0-7 days | Schedule baseline CAPS-5. |
| | follow-up | following end | |
| | | of medication | |
| | | taper | |
| Baseline and | Baseline | Following | Complete CAPS-5, Sheehan Disability Score |
| Enrollment | Assessments | Preparatory | (SDS), and Dissociative Subtype of PTSD |
| Confirmation | | Session 2 | Scale (DSPS) by IR via in-person or |
| | | | telemedicine appointment. Scores forwarded to |
| | | | therapy monitoring team. Resumption of |
| | | | tapered medicine in symptom management |
| | | | requires. Withdrawal of participants not |
| | | | meeting eligibility criteria at this point. |
| | Preparatory | 1-7 days | Participants complete baseline self-report |
| | Session 3 | following | metrics and schedule Experimental Session 1. |
| | | baseline | |
| | | CAPS-5 | |

The Treatment Period schedule follows the Screening Period and the Preparatory Period

Treatment Period (lasts approximately 12 weeks)

| Study Visit | | Visit Timing | Description |
|---|---|---|---|
| Treatment | Randomization | 0-10 days | Complete following verification participant |
| 1 | | following | is still enrolled and Experimental Session 1 |
| | | Baseline | is scheduled. Double-blind randomization. |
| | | assessments | |
| | Experimental | 8 hours plus | Patient's weight is determined for dosage |
| | Session 1 | overnight | calculation. Baseline STAI assessment. |
| | | observation | Dose is 180-210 mg p.o. Placebo |
| | | | administered in placebo group at same time |
| | | | interval. |
| | | Optional. | REMS Protocol: Considered for |
| | | Following | administration of racemic MBDB, R- |
| | | anxiety | MBDB, and S-MBDB treatment groups and |
| | | assessment 0.75- | associated placebo controls. Underdosing of |
| | | 2 hours after | racemic MBDB, R-MBDB, or S-MBDB |
| | | first dose | may lead to exacerbation of anxiogenic |
| | | administration | features necessitating careful assessment of |
| | | | worsening of symptoms. Patients are |
| | | | assessed for anxiety about 0.75 hours after |
| | | | first dose. With no change or increase in |
| | | | anxiety, patients are given a dose of 30%- |
| | | | 50% of first dose and anxiety reassessed in |
| | | | about 1 hour. Dose titration up to a |
| | | | maximum cumulative dose of 350 mg. |
| | | | Placebo administered in placebo group at |
| | | | same time interval. If anxiety is decreased, |
| | | | no REMS protocol dose is given at this time. |
| | | 1.5-2 hours | Supplemental Dose: Supplemental half-dose |
| | | after first dose | of 50-100 mg administered 1.5-2 hours |
| | | administration | following initial dose administration unless |
| | | | contraindicated. If initial plus REMS |
| | | | protocol doses total a cumulative dose of |
| | | | 350 mg, no Supplemental Dose is given. If |
| | | | initial plus any REMS protocol dose is less |
| | | | than 350 mg, Supplemental Dose is |
| | | | administered 1.5 to 2 hours following initial |
| | | | dose administration. Placebo administered in |
| | | | placebo group at same time intervals. |
| | Intervening | Morning | Behavioral or cognitive-behavioral therapy |
| | Session 1A | following | session lasting 90-120 minutes. Assessment |
| | | Experimental | of potential anxiogenic, mixed anxiogenic- |
| | | Session 1 | anxiolytic, or anxiolytic treatment effects. |
| | | | CAPS-5 assessment. Instructions for |
| | | | participants to complete C-SSRS |
| | | | assessments on every two days following |
| | | | Experimental Session 1. |
| | Intervening | 3 to 14 days | Behavioral or cognitive-behavioral therapy |
| | Session 1B | following | session lasting 60-120 minutes. CAPS-5 |
| | | Experimental | assessment. |
| | | Session 1 | |
| | Intervening | 18-34 days | Behavioral or cognitive-behavioral therapy |
| | Session 1C | following | session lasting 60-120 minutes. |
| | | Experimental | Assessments include LEC-5, HAM-A, C- |
| | | Session 1 | SSRS and CAPS-5. |
| Treatment | Experimental | 8 hours plus | Patient's weight is determined for dosage |
| 2 | Session 2 | overnight | calculation. Baseline STAI assessment. |
| | | observation. 19- | Dose is 180-210 mg p.o. Placebo |
| | | 35 days | administered in placebo group at same time |
| | | following | interval. |
| | | Experimental | |
| | | Session 1. | |
| | | Optional. | REMS Protocol: Considered for |
| | | Following | administration of racemic MBDB, R- |
| | | anxiety | MBDB, and S-MBDB treatment groups and |
| | | assessment 0.75- | associated placebo controls. Underdosing of |
| | | 2 hours after | racemic MBDB, R-MBDB, or S-MBDB |
| | | first dose | may lead to exacerbation of anxiogenic |
| | | administration | features necessitating careful assessment of |
| | | | worsening of symptoms. Patients are |
| | | | assessed for anxiety about 0.75 hours after |
| | | | first dose. With no change or increase in |
| | | | anxiety, patients are given a dose of 30%- |
| | | | 50% of first dose and anxiety reassessed in |
| | | | about 1 hour. Dose titration up to a |
| | | | maximum cumulative dose of 350 mg. |
| | | | Placebo administered in placebo group at |
| | | | same time interval. If anxiety is decreased, |
| | | | no REMS protocol dose is given at this time. |
| | | 1.5-2 hours | Supplemental Dose: Supplemental half-dose |
| | | after first dose | of 50-100 mg administered 1.5 to 2 hours |
| | | administration | following initial dose administration unless |
| | | | contraindicated. If initial plus REMS |
| | | | protocol doses total a cumulative dose of |
| | | | 350 mg, no Supplemental Dose is given. If |
| | | | initial plus any REMS protocol dose is less |
| | | | than 350 mg, Supplemental Dose is |
| | | | administered 1.5 to 2 hours following initial |
| | | | dose administration. Placebo administered in |
| | | | placebo group at same time intervals. |
| | Intervening | Morning | Behavioral or cognitive-behavioral therapy |
| | Session 2A | following | session lasting 90-120 minutes. |
| | | Experimental | Assessment of potential anxiogenic, mixed |
| | | Session 2 | anxiogenic-anxiolytic, or anxiolytic |
| | | | treatment effects. CAPS-5 assessment. |
| | | | Instructions for participants to complete C- |
| | | | SSRS assessments on every two days |
| | | | following Experimental Session 1. |
| | Intervening | 3 to 14 days | Behavioral or cognitive-behavioral therapy |
| | Session 2B | following | session lasting 60-120 minutes. CAPS-5 |
| | | Experimental | assessment. |
| | | Session 2 | |
| | Intervening | 18-34 days | Behavioral or cognitive-behavioral therapy |
| | Session 2C | following | session lasting 60-120 minutes. |
| | | Experimental | Assessments include LEC-5, HAM-A, and |
| | | Session 2 | C-SSRS and CAPS-5.. |
| Treatment | Experimental | 8 hours plus | Patient's weight is determined for dosage |
| 3 | Session 3 | overnight | calculation. Baseline STAI assessment. |
| | | observation. 19- | Dose is 180-210 mg p.o. Placebo |
| | | 35 days | administered in placebo group at same time |
| | | following | interval. |
| | | Experimental | |
| | | Session 2. | |
| | | Optional. | REMS Protocol: Only considered for |
| | | Following | administration of racemic MBDB and S- |
| | | anxiety | MBDB treatment groups and associated |
| | | assessment 0.75- | placebo controls. Underdosing of racemic |
| | | 2 hours after | MBDB or S-MBDB may lead to |
| | | first dose | exacerbation of anxiogenic features |
| | | administration | necessitating careful assessment of |
| | | | worsening of symptoms. Patients are |
| | | | assessed for anxiety about 0.75 hours after |
| | | | first dose. With no change or increase in |
| | | | anxiety, patients are given a dose of 30%- |
| | | | 50% of first dose and anxiety reassessed in |
| | | | about 1 hour. Dose titration up to a |
| | | | maximum cumulative dose of 350 mg. |
| | | | Placebo administered in placebo group at |
| | | | same time interval. If anxiety is decreased, |
| | | | no REMS protocol dose is given at this time. |
| | | 1.5-2 hours | Supplemental Dose: Supplemental half-dose |
| | | after first dose | of 50-100 mg administered 1.5 to 2. hours |
| | | administration | following initial dose administration unless |
| | | | contraindicated. If initial plus REMS |
| | | | protocol doses total a cumulative dose of |
| | | | 350 mg, no Supplemental Dose is given. If |
| | | | initial plus any REMS protocol dose is less |
| | | | than 350 mg, Supplemental Dose is |
| | | | administered 2 to 2.5 hours following initial |
| | | | dose administration. Placebo administered in |
| | | | placebo group at same time intervals. |
| | Intervening | Morning | Behavioral or cognitive-behavioral therapy |
| | Session 3A | following | session lasting 90-120 minutes. |
| | | Experimental | Assessment of potential anxiogenic, mixed |
| | | Session 3 | anxiogenic-anxiolytic, or anxiolytic |
| | | | treatment effects. CAPS-5 assessment. |
| | | | Instructions for participants to complete C- |
| | | | SSRS assessments on every two days |
| | | | following Experimental Session 1. |
| | Intervening | 3 to 14 days | Behavioral or cognitive-behavioral therapy |
| | Session 3B | following | session lasting 60-120 minutes. CAPS-5 |
| | | Experimental | assessment. |
| | | Session 3 | |
| | Intervening | 18-34 days | Behavioral or cognitive-behavioral therapy |
| | Session 3C | following | session lasting 60-120 minutes. |
| | | Experimental | Assessments include LEC-5, HAM-A, C- |
| | | Session 3 | SSRS and CAPS-5. |

The Follow-up Period schedule and Study Conclusion follow the Screening Period and the Treatment Period.

Follow-up Period and Study Conclusion

| Study Visit | Visit Timing | Description |
|---|---|---|
| Follow-up Period | Occurs 2-10 days | Occurs about 100-150 days following Baseline |
| | after Intervening | assessment. Complete self-reported |
| | Session 3C. | assessments and patient safety measures. |
| | | Create exit treatment plan for participant based |
| | | on results. Final CAPS-5 assessment. Final |
| | | SDS, HAM-D, and ESS assessments. |
| Study Conclusion | At time of | Inform participants who finished study |
| | unblinding of group. | protocol of unblinding of groups. If a |
| | | participant was in a placebo group, offer |
| | | opportunity to enroll in a open-label safety |
| | | extension study using either racemic MBDB, |
| | | S-MBDB, or R-MBDB. |

### Dose Selection

This study compares the effects of three blinded Experimental Sessions of psychotherapy in combination with flexible doses of MBDB or placebo administered as described below. Non-drug preparatory and intervening psychotherapy sessions are also included. Patient's weight is determined for dosage calculation. Initial dose is 180 mg unless this will result in a dosage of less than 1.5 mg/kg of patient weight. Initial dose thereby adjusted upward in 50 mg increments to deliver the lowest dose possible of at least 1.5 mg/kg of patient weight. Initial dose for Experimental Session 2 and 3 is cumulative dose calculated by adding the initial dose plus REMS protocol dose used the previous Experimental Session for each patient.

| Double- | | | Optional (REMS) | | |
|---|---|---|---|---|---|
| blinded | | | protocol: Dose | Supplemental | |
| treatment | Experimental | Initial | Titration if | Dose (unless | Cumulative |
| group | Session | Dose | underdosing occurs | contraindicated) | Dose |
| Racemic | 1 | 180-210 mg | 30-100 mg | 50-100 mg | 180-350 mg |
| MBDB | | | | | |
| Racemic | 2 | 180-210 mg | 30-100 mg | 50-100 mg | 180-350 mg |
| MBDB | | | | | |
| Racemic | 3 | 180-210 mg | 30-100 mg | 50-100 mg | 180-350 mg |
| MBDB | | | | | |
| S-MBDB | 1 | 180-210 mg | 30-100 mg | 50-100 mg | 180-350 mg |
| S-MBDB | 2 | 180-210 mg | 30-100 mg | 50-100 mg | 180-350 mg |
| S-MBDB | 3 | 180-210 mg | 30-100 mg | 50-100 mg | 180-350 mg |
| R-MBDB | 1 | 180-210 mg | 30-100 mg | 50-100 mg | 180-350 mg |
| R-MBDB | 2 | 180-210 mg | 30-100 mg | 50-100 mg | 180-350 mg |
| R-MBDB | 3 | 180-210 mg | 30-100 mg | 50-100 mg | 180-350 mg |

### Randomization and Masking

Randomization occurs prior to the initiation of Experimental Session 1. Each participant is provided the next randomized number in a sequence by a blinded study monitor. Participants are then randomized, according to a computer-generated randomization schedule, 1:1:1:1 to racemic MBDB, S-MBDB, R-MBDB, or placebo. The randomization schedule is prepared and implemented by an independent statistician. Participants, clinicians, and study teams are blinded to treatment allocation. Racemic MBDB, R-MBDB, and S-MBDB treatment groups may be subjected to anxiogenic effects due to underdosing of participants. As such, an optional dose titration schedule (REMS protocol) exists for racemic MBDB, R-MBDB, and S-MBDB treatment groups if a participant displays no change or a significant worsening of assessed anxiety symptomatology. Participants are assessed for general well-being and anxiety by a medical practitioner about 0.75 hours after the first dose is administered. Assessments performed may include general assessments of physical and mental well-being, a structured clinical interview for DSM-5 (SCID-5) module A1, and/or a STAI assessment and may continue throughout the period of overnight observation.

Subjects then undergo three Intervening Sessions with the first session the morning after the initial dose administration. MBDB treatment group or placebo group participants qualifying with a significant worsening of assessed anxiety symptomatology would undergo a placebo dose titration administration. Subjects would then undergo three Intervening Sessions with the first session the morning after the placebo dose titration administration. The pharmacist at each site, who prepares the treatments according to the randomization schedule, and an unblinded monitor, who performs drug accountability during the study, are unblinded. No other study personnel are unblinded until after formal locking of the study database. In the event of a medical emergency, the pharmacist is to reveal actual treatment contents to the primary investigator, who is to alert the Sponsor of the emergency. If the participant or study center personnel are unblinded, the subject is to be removed from the study.

### Outcomes

The primary objective of this study is to evaluate the efficacy and safety of MBDB treatment combined with psychotherapy to treat moderate to severe PTSD compared to identical psychotherapy combined with placebo treatment. MBDB treatment is further subdivided into three separate treatment groups (racemic MBDB, S-MBDB, and R-MBDB) with each treatment subgroup only receiving administration of the single assigned drug. Treatment outcomes are determined based on a change in CAPS-5 Total Severity.

Several secondary objectives are designed for this study. One is an evaluation of clinician-rated functional impairment of MBDB treatment combined with psychotherapy to treat moderate to severe PTSD compared to identical psychotherapy combined with placebo treatment. MBDB treatment is further subdivided into three separate treatment groups (racemic MBDB, S-MBDB, and R-MBDB) with each treatment subgroup only receiving administration of the single assigned drug. Treatment outcomes are determined based on a change in SDS. Another secondary objective of this study is to evaluate clinician-rated depression of MBDB treatment combined with psychotherapy to treat moderate to severe PTSD compared to identical psychotherapy combined with placebo treatment. Identical study parameters are in place as for the clinician-rated functional impairment assessment except that treatment outcomes are determined based on a change in HAM-D. An additional secondary objective of this study is to evaluate sleep assessments of MBDB treatment combined with psychotherapy to treat moderate to severe PTSD compared to identical psychotherapy combined with placebo treatment. Identical study parameters are in place as for the clinician-rated functional impairment assessment except that treatment outcomes are determined based on a change in ESS. Co-morbidities present in participants with a strong positive response to MBDB treatment are correlated. Co-morbidities present in participants with weak-to-no positive response to MBDB treatment are correlated. Changes to presence or severity of co-morbidities from the Preparation Period to the Study Conclusion are recorded to determine if MBDB treatment combined with psychotherapy in moderate to severe PTSD subjects affects co-morbid phenotypes not falling under the constellation of PTSD symptoms.

### Participant Populations

Participants are recruited through referrals by other treatment providers or through print or internet advertisements. The Sponsor monitors demographics of individuals assessed for enrollment to encourage diversity and an unbiased representation of the total PTSD population. Participants must be 18 years of age or older, have a confirmed diagnosis of at least moderate PTSD according to PCL-5 at the Screening Period. Medical history intake must indicate a presence of PTSD symptoms for at least 6 months prior to the Screening Period. Participants may be enrolled in the study while remaining on a treatment regimen involving SSRI or SNRI treatment prescribed for PTSD. In some cases, enrolled participants currently taking an SSRI, an SNRI, or another medication are tapered off these medications and stabilized prior to baseline assessments. Participants with a confirmed personality disorder diagnosis are excluded from this study. Participants must be in good general physical health without one or more severe chronic conditions that could affect the safety or tolerability of MBDB treatment.

### Statistical Analysis

The change from baseline in CAPS-5, SDS, HAM-D, and ESS in participants is analyzed using a mixed effects model for repeated measures (MMRM) to obtain covariance parameter estimates. The model includes treatment center, treatment subtype, baseline assessments, assessment time point, and time point-by-treatment as explanatory variables. Treatment center is treated as a random effect; all other explanatory variables are treated as fixed effects. Model-based point estimates (e.g., least squares means, 95% confidence intervals, and p-values) are reported for each time point. With a sample size of 50 participants per treatment group, this study has 90% power to detect a significant treatment effect, using a two-sided test with an alpha value of 0.05. Additional participants may be enrolled with conditional power analysis conducted at a group-unblinded interim analysis time point for efficacy when 200 participants are enrolled and at least 60% of the blinded participants (N=120) have completed a final CAPS-5 assessment and reached Study Conclusion.

### Results

The results may indicate that the primary objective is achieved. At the point of Study Conclusion, racemic MBDB-treated, S-MBDB-treated, and R-MBDB-treated participants may demonstrate a significant mean reduction in CAPS-5 assessment compared to the placebo group. The S-MBDB-treated subgroup may achieve a significant mean reduction in CAPS-5 assessment with a lower total dosage of drug compared to the racemic MBDB-treated subgroup. The R-MBDB-treated subgroup may achieve a significant mean reduction in CAPS-5 assessment with a lower total dosage of drug compared to the racemic MBDB-treated subgroup. Significant improvements in CAPS-5 assessments may be observed for racemic MBDB-treated, S-MBDB-treated, and R-MBDB-treated participants at time points of Intervening Session 1C, Intervening Session 2C, Intervening Session 3C and Study Conclusion, compared to placebo-treated controls. Significant improvements in CAPS-5 assessments may be observed for S-MBDB-treated participants at time points of Intervening Session 1C, Intervening Session 2C, Intervening Session 3C, compared to placebo-treated controls without a significant increase in adverse anxiogenic incidents in S-MBDB-treated participants.

The results may indicate that the secondary objectives of this study are also achieved. Racemic MBDB-treated, S-MBDB-treated, and R-MBDB-treated participants may demonstrate a significant improvement in clinician-rated functional impairment score as measured by SDS compared to placebo-treated controls. Racemic MBDB-treated, S-MBDB-treated, and R-MBDB-treated participants may demonstrate a significant improvement depression as measured by HAM-D compared to placebo-treated controls. Racemic MBDB-treated, S-MBDB-treated, and R-MBDB-treated participants may demonstrate a significant improvement in lessening daytime sleepiness as measured by ESS. S-MBDB-treated participants may demonstrate a significant improvement in clinician-rated functional impairment score, in depression, and in lessening daytime sleepiness compared to placebo-treated controls without a significant increase in adverse anxiogenic incidents in S-MBDB-treated participants.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. An isotopically enriched compound of the formula 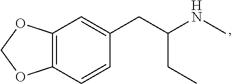
or a pharmaceutically acceptable salt thereof.

2. The compound of claim 1, having the formula 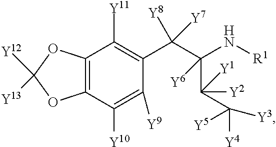
wherein
at least one of R¹, Y¹, Y², Y³, Y⁴, Y⁵, Y⁶, Y⁷, Y⁸, Y⁹, Y¹⁰, Y¹¹, Y¹² and Y¹³ is enriched in at least one heavy isotope, or a pharmaceutically acceptable salt thereof.

3. The compound of claim 2, wherein the heavy isotope is selected from ¹⁴C, tritium and deuterium.

4. The compound of claim 2, wherein R¹ is selected from CD₃, CD₂H, CDH₂, CT₃, CT₂H, CTH₂ and CH₃; and
Y¹, Y², Y³, Y⁴, Y⁵, Y⁶, Y⁷, Y⁸, Y⁹, Y¹⁰, Y¹¹, Y¹² and Y¹³ are each independently selected from protium, deuterium and tritium.

5. The compound of claim 2, wherein at least one of R, Y¹, Y², Y³, Y⁴, Y⁵, Y⁶, Y⁷, Y⁸, Y⁹, Y¹⁰, Y¹¹, Y¹² and Y¹³ is enriched in deuterium.

6. The compound of claim 1, wherein the compound is selected from the group consisting of 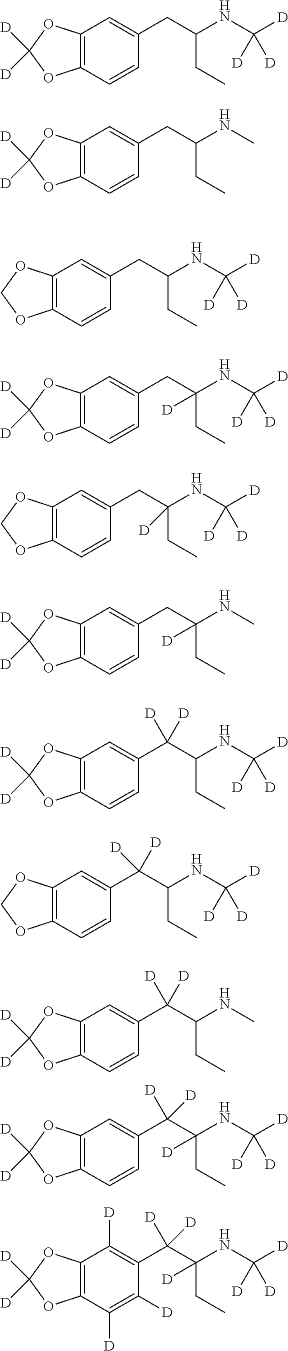 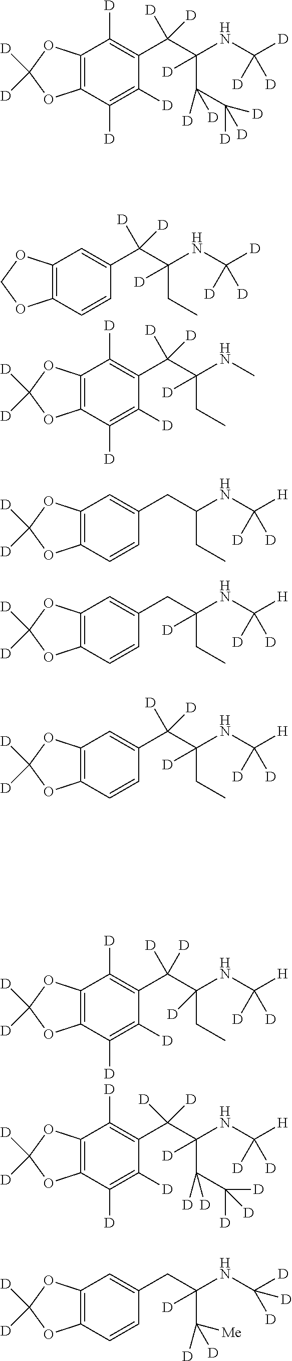 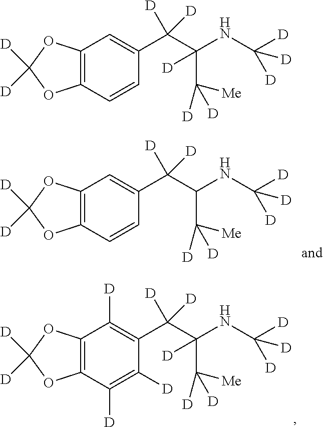
or a pharmaceutically acceptable salt thereof.

7. The compound of claim 1, having the formula

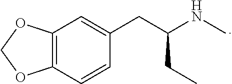

8. The compound of claim 1, having the formula

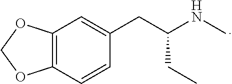

9. The compound of claim 7, having the formula 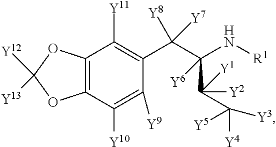
wherein
at least one of R¹, Y¹, Y², Y³, Y⁴, Y⁵, Y⁶, Y⁷, Y⁸, Y⁹, Y¹⁰, Y¹¹, Y¹² and Y¹³ is enriched in at least one heavy isotope, or a pharmaceutically acceptable salt thereof.

10. The compound of claim 8, having the formula 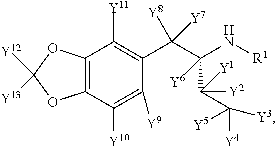
wherein
at least one of R¹, Y¹, Y², Y³, Y⁴, Y⁵, Y⁶, Y⁷, Y⁸, Y⁹, Y¹⁰, Y¹¹, Y¹² and Y¹³ is enriched in at least one heavy isotope, or a pharmaceutically acceptable salt thereof.

11. The compound of claim 9, selected from the group consisting of 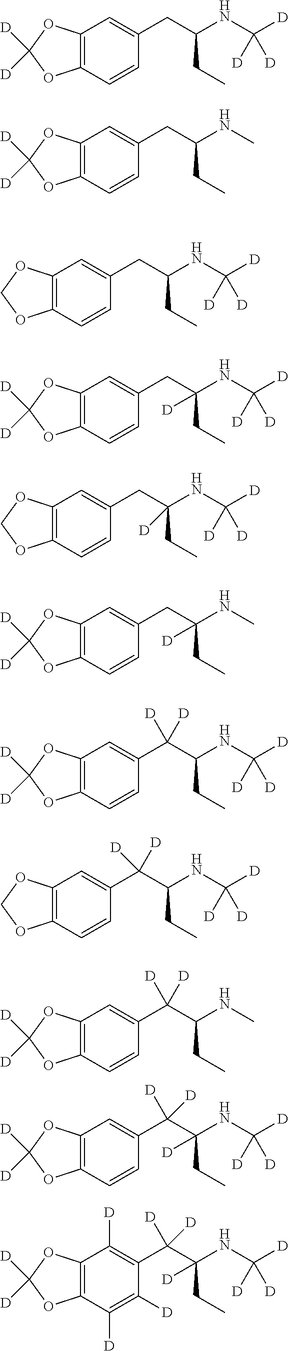 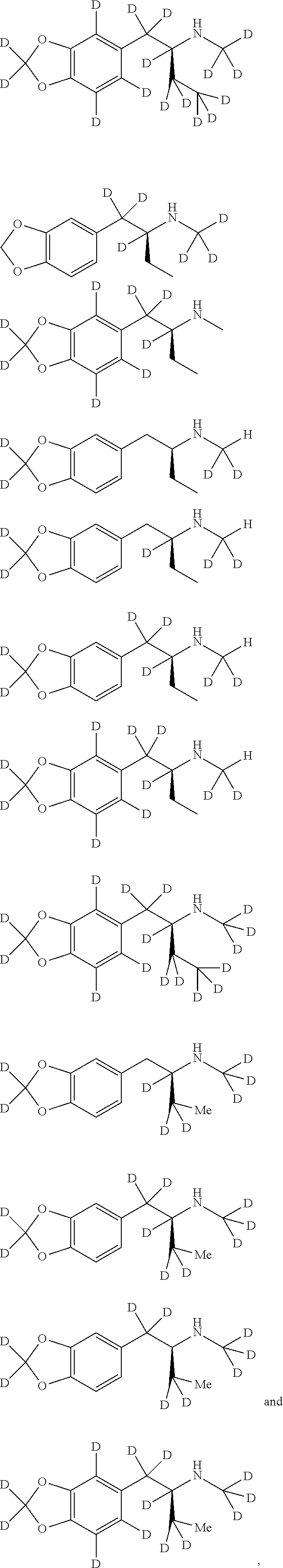
or a pharmaceutically acceptable salt thereof.

12. The compound of claim 10, selected from the group consisting of 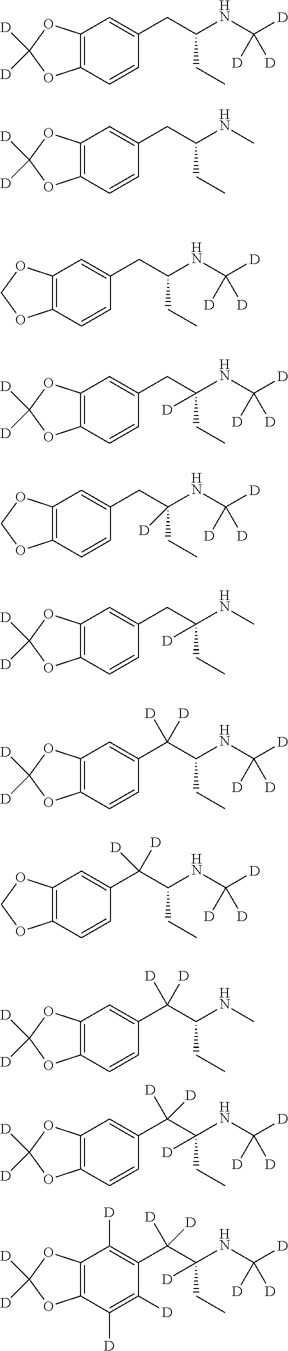 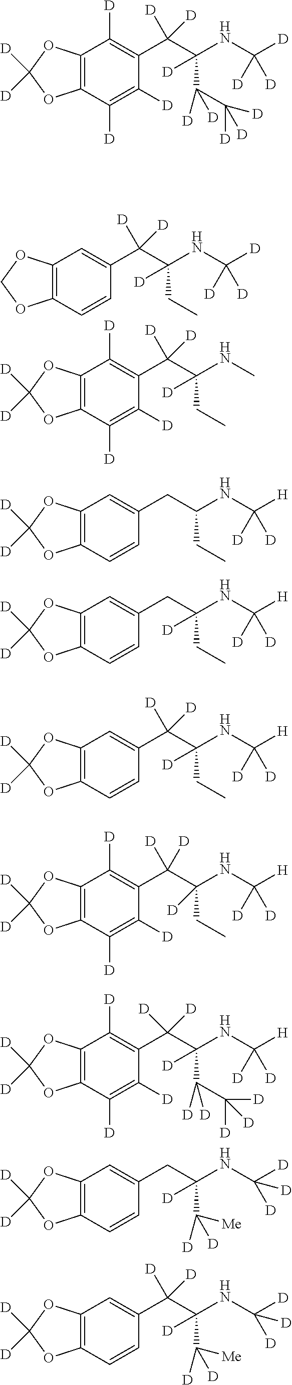 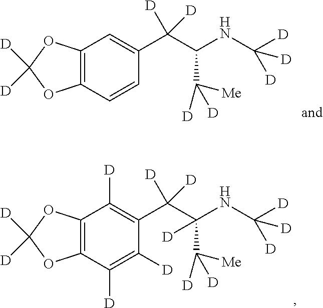 [SMILES: [2H]C([2H])([2H])N[C@H](C([2H])([2H])C)C([2H])([2H])c1ccc2c(c1)OC([2H])([2H])O2 ; [2H]c1c([2H])c(C([2H])([2H])[C@]([2H])(NC([2H])([2H])[2H])C([2H])([2H])C)c([2H])c2c1OC([2H])([2H])O2]
or a pharmaceutically acceptable salt thereof.

13. A compound having the structure of any one of the compounds shown in Table 1 or a pharmaceutically acceptable salt thereof.

14. The isotopically enriched compound of claim 1, wherein the isotopically enriched compound is in the form of a pharmaceutically acceptable salt.

15. The isotopically enriched compound of claim 1, wherein the isotopically enriched compound is in the form of a solvate.

16. A pharmaceutical composition comprising a compound of claim 1.

17. A method for increasing neuronal plasticity, comprising contacting a neuron with an effective amount of the compound of claim 1.

18. (canceled)

19. A method for treating a neurological disorder or a psychiatric disorder, or both, in a subject in need thereof, comprising administering an effective amount of the compound of claim 1 or a pharmaceutical composition thereof to the subject.

20.-24. (canceled)

25. A method of treating an anxiety disorder or depressive disorder in a subject in need thereof, wherein the method comprises administering to the subject an effective amount of the compound of claim 7, or a deuterated form thereof.

26.-27. (canceled)

28. A method of treating an anxiety or depressive disorder in a subject in need thereof, the method comprising administering to the subject an effective amount of S-MBDB.

29.-33. (canceled)
